(19) United States
(12) Patent Application Publication
(10) Pub. No.: US 2025/0163505 A1
(43) Pub. Date: May 22, 2025

(54) CONJUGATED POLYMERS OR POLYMER DOTS AS FLUORESCENT LABELS

(71) Applicant: Illumina, Inc., San Diego, CA (US)

(72) Inventors: Nam Nguyen, Cambridge (GB); Iuliana Petruta Maria, Cambridge (GB); Andrew A. Brown, Cambridge (GB); Wayne N. George, Cambridge (GB); Xiaolin Wu, Cambridge (GB); Xavier Von Hatten, Cambridge (GB); Madushani Dharmarwardana, Cambridge (GB)

(21) Appl. No.: 18/947,955

(22) Filed: Nov. 14, 2024

Related U.S. Application Data
(60) Provisional application No. 63/600,417, filed on Nov. 17, 2023.

(51) Int. Cl. C12Q 1/6874; C08F 134/04; G01N 33/58

(52) U.S. Cl. CPC C12Q 1/6874; C08F 134/04; G01N 33/582

(57) ABSTRACT

Embodiments of the present disclosure relate to compositions and methods for labeling of nucleotides, oligonucleotides, or polynucleotides with conjugated polymers or polymer dots. In particular, conjugated polymers or polymer dots can be used as detectable labels for nucleotides, oligonucleotides, or polynucleotides in sequencing by synthesis.

### CROSS-REFERENCE TO RELATED APPLICATIONS

The present application claims the benefit of priority to U.S. provisional application No. 63/600,417, filed Nov. 17, 2023, the content of which is incorporated by reference in its entirety.

### FIELD

The present disclosure generally relates to compositions, kits, methods and systems for nucleic acid sequencing applications.

### BACKGROUND

Nucleic acid sequencing methodology has evolved significantly from the chemical degradation methods used by Maxam and Gilbert and the strand elongation methods used by Sanger. Today several sequencing methodologies are in use which allow for the parallel processing of thousands of nucleic acids all in a single sequencing run. The instrumentation that performs such methods is typically large and expensive since the current methods typically rely on large amounts of expensive reagents and multiple sets of optic filters to record nucleic acid incorporation into sequencing reactions.

It has become clear that the need for high-throughput, smaller, less expensive DNA sequencing technologies will be beneficial for reaping the rewards of genome sequencing. Personalized healthcare is moving toward the forefront and will benefit from such technologies. The sequencing of an individual's genome to identify potential mutations and abnormalities will be crucial in identifying if a person has a particular disease, followed by subsequent therapies tailored to that individual. To accommodate such endeavor, sequencing technologies should not only have high throughput capabilities, but also have scalability. As such, there exists a need for new sequencing methods that improve on speed, error read, and are also cost effective.

### SUMMARY

The present disclosure provides next-generation sequencing kits, methods, systems, and compositions.

One aspect of the present disclosure relates to a labeled nucleotide, oligonucleotide or polynucleotide comprising a water-soluble conjugated polymer or a water-dispersible polymer dot attached to the nucleotide, oligonucleotide or polynucleotide as a detectable label. In some embodiments, the water-soluble conjugated polymer or a water-dispersible polymer dot attached to the nucleotide, oligonucleotide or polynucleotide via covalent bonding.

In some embodiments, the water-soluble conjugated polymer comprises one or more repeating unit moieties selected from the group consisting of:

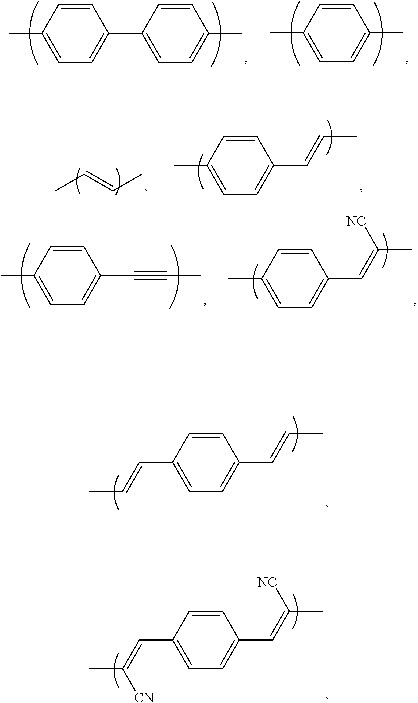

a five membered heteroarylene containing one, two or three heteroatoms selected from the group consisting of N, O and S,

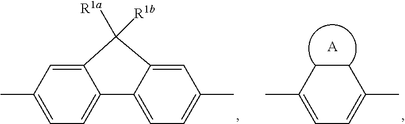

and combinations thereof;
wherein each of R^{1a}, R^{1b}, R^{1c}, R^{1d}, R^{1e} and R^{1f} is independently H, unsubstituted C₁-C₁₂ alkyl, C₁-C₁₂ alkyl substituted with one or more water solubilizing groups, or 2 to 20 membered heteroalkyl;
ring A is a five or six membered heteroaryl containing one, two or three heteroatoms selected from the group consisting of N, O and S, and
wherein each structure is optionally substituted with one or more electron donating groups, one or more electron withdrawing groups, one or more functional moiety R^{F}, or one or more water solubilizing group R^{S}, or combinations thereof.

In some embodiments, the water-dispersible polymer dot comprises a conjugated polymer nanoparticle and one or more functionalized surfactants attached to the surface of the nanoparticle through non-covalent interaction.

In some other embodiments, the water-dispersible polymer dot comprises a core-shell water-dispersible polymer dot attached thereto the nucleotide (e.g., via covalent bonding or non-covalent interactions), oligonucleotide or polynucleotide, wherein the core-shell water-dispersible polymer dot comprises a conjugated polymer nanoparticle core, and a hydrogel shell. In some other embodiments, the core-shell water-dispersible polymer dot comprises a water-soluble conjugated polymer coated on a supporting particle matrix core. In other embodiments, the conjugated polymer may be a hydrophilic polymer or a water-swellable polymer.

In some further embodiments, the water-soluble conjugated polymer or the water-dispersible polymer dot is covalently bounded to an unlabeled nucleotide, or an unlabeled nucleotide moiety at the terminal (e.g., 3′ terminal) of the oligonucleotide or polynucleotide.

Another aspect of the present disclosure relates to a kit for sequencing application, comprising:
an incorporation mixture composition comprising one or more of four different types of nucleotides each comprising a 3′ blocking group, wherein a first type of unlabeled nucleotide comprises a first reactive moiety covalently attached thereto; and
a first labeling reagent comprising a water-soluble conjugated polymer or a water-dispersible polymer dot, each having a first functional moiety that is capable of reacting specifically with the first reactive moiety of the first type of unlabeled nucleotide to form covalent or non-covalent bonding.

Another aspect of the present disclosure relates to a kit for sequencing application, comprising: an incorporation mixture composition comprising one or more of four different types of nucleotides each comprising a 3′ blocking group, wherein a first type of nucleotide is the labeled nucleotide described herein.

Another aspect of the present disclosure relates to a method of determining the sequences of a plurality of different target polynucleotides, comprising:
(a) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(b) contacting the solid support with an aqueous solution comprising DNA polymerase and one or more of four different types of unlabeled nucleotides under conditions suitable for DNA polymerase-mediated primer extension, and incorporating one type of nucleotides into the sequencing primers to produce extended copy polynucleotides; wherein
each of the four types of nucleotides comprises a 3′ blocking group; and
the incorporation mixture comprises a first type of unlabeled nucleotide having a first reactive moiety covalently attached to the first type of unlabeled nucleotide;
(c) contacting the extended copy polynucleotides with a first labeling reagent comprising a water-soluble conjugated polymer or a water-dispersible polymer dot comprising a functional moiety that reacts specifically with the first reactive moiety of the first type of unlabeled nucleotides to form covalent or non-covalent bonding;
(d) imaging the solid support and performing one or more fluorescent measurements of the extended copy polynucleotides; and
(e) removing the 3′ blocking group of the incorporated nucleotides.

### BRIEF DESCRIPTION OF THE DRAWINGS

FIG. 1 schematically illustrates a step of the sequencing by synthesis in which an unlabeled nucleotide is incorporated into a growing polynucleotide chain followed by post incorporation labeling of a labeling reagent.

FIG. 2A schematically illustrates a functionalized water-soluble conjugated polymer according to an embodiment of the present disclosure.

FIG. 2B schematically illustrates a stabilized water-dispersible polymer dot according to an embodiment of the present disclosure.

FIG. 2C schematically illustrates a core-shell water-dispersible polymer dot where a core of the polymer dot includes a conjugated polymer according to an embodiment of the present disclosure.

FIG. 2D schematically illustrates a core-shell water-dispersible polymer dot where a shell of the polymer dot includes a conjugated polymer according to an embodiment of the present disclosure.

FIG. 3 illustrates a process of synthesizing a stabilized water-dispersible polymer dot including stabilizers according to an embodiment of the present disclosure.

FIG. 4 schematically illustrates a process of synthesizing a core-shell polymer dot where a conjugated polymer makes up a core of the polymer dot, according to an embodiment of the present disclosure.

FIG. 5 schematically illustrates an example process of synthesizing a polymer dot where the core is a supporting matrix and the conjugated polymer makes up a shell of the polymer dot, according to an embodiment of the present disclosure.

FIGS. 6A and 6B are scatterplots of two-channel SBS runs. FIG. 6A was conducted with standard two-channel SBS reagents (i.e., with pre-labeled fully functionalized nucleotides). FIG. 6B was conducted with a set of unlabeled fully functionalized C nucleotides (ffCs) having a TCO reactant moiety and a methyl tetrazine functionalized polymer dot capable of binding to the reactant moiety of the ffCs.

### DETAILED DESCRIPTION

### Definitions

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps but may include additional steps. When used in the context of a compound, composition, or device, the term “comprising” means that the compound, composition, or device includes at least the recited features or components, but may also include additional features or components.

As used herein, common organic abbreviations are defined as follows:
° C. Temperature in degrees Centigrade
dATP Deoxyadenosine triphosphate
dCTP Deoxycytidine triphosphate
dGTP Deoxyguanosine triphosphate
dTTP Deoxythymidine triphosphate
ddNTP Dideoxynucleotide triphosphate
ffA Fully functionalized A nucleotide
ffC Fully functionalized C nucleotide
ffG Fully functionalized G nucleotide
ffN Fully functionalized nucleotide
ffT Fully functionalized T nucleotide
LED Light emitting diode
SBS Sequencing by synthesis

As used herein, the term “array” refers to a population of different probe molecules that are attached to one or more substrates such that the different probe molecules can be differentiated from each other according to relative location. An array can include different probe molecules that are each located at a different addressable location on a substrate. Alternatively, or additionally, an array can include separate substrates each bearing a different probe molecule, wherein the different probe molecules can be identified according to the locations of the substrates on a surface to which the substrates are attached or according to the locations of the substrates in a liquid. Exemplary arrays in which separate substrates are located on a surface include, without limitation, those including beads in wells as described, for example, in U.S. Pat. No. 6,355,431 B1, US 2002/0102578 and PCT Publication No. WO 00/63437. Exemplary formats that can be used in the invention to distinguish beads in a liquid array, for example, using a microfluidic device, such as a fluorescent activated cell sorter (FACS), are described, for example, in U.S. Pat. No. 6,524,793. Further examples of arrays that can be used in the invention include, without limitation, those described in U.S. Pat. Nos. 5,429,807; 5,436,327; 5,561,071; 5,583,211; 5,658,734; 5,837,858; 5,874,219; 5,919,523; 6,136,269; 6,287,768; 6,287,776; 6,288,220; 6,297,006; 6,291,193; 6,346,413; 6,416,949; 6,482,591; 6,514,751 and 6,610,482; and WO 93/17126; WO 95/11995; WO 95/35505; EP 742 287; and EP 799 897.

As used herein, the term “covalently attached” or “covalently bonded” refers to the forming of a chemical bonding that is characterized by the sharing of pairs of electrons between atoms. For example, a covalently attached polymer coating refers to a polymer coating that forms chemical bonds with a functionalized surface of a substrate, as compared to attachment to the surface via other means, for example, adhesion or electrostatic interaction. It will be appreciated that polymers that are attached covalently to a surface can also be bonded via means in addition to covalent attachment.

As used herein, the term “non-covalent bonding” or “non-covalent interactions” differs from a covalent bond in that it does not involve the sharing of electrons, but rather involves more dispersed variations of electromagnetic interactions between molecules or within a molecule. Non-covalent interactions can be generally classified into four categories, electrostatic, π-effects, van der Waals forces, and hydrophobic effects. Non-limiting examples of electrostatic interactions include ionic interactions, hydrogen bonding (a specific type of dipole-dipole interaction), halogen bonding, etc. Van der Walls forces are a subset of electrostatic interaction involving permanent or induced dipoles or multipoles. π-effects can be broken down into numerous categories, including (but not limited to) π-π interactions, cation-π & anion-π interactions, and polar-π interactions. In general, π-effects are associated with the interactions of molecules with the π-orbitals of a molecular system, such as benzene. The hydrophobic effect is the tendency of nonpolar substances to aggregate in aqueous solution and exclude water molecules. Non-covalent interactions can be both intermolecular and intramolecular. Non-covalent interactions can be both intermolecular and intramolecular.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “Cₐ to C_{b},” “Cₐ-C_{b},” or “C_{a-b}” in which “a” and “b” are integers refer to the number of carbon atoms in an alkyl, alkenyl or alkynyl group, or the number of ring atoms of a cycloalkyl or aryl group. That is, the alkyl, the alkenyl, the alkynyl, the ring of the cycloalkyl, and ring of the aryl can contain from “a” to “b,” inclusive, carbon atoms. For example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—; a C₃ to C₄ cycloalkyl group refers to all cycloalkyl groups having from 3 to 4 carbon atoms, that is, cyclopropyl and cyclobutyl. Similarly, a “4 to 6 membered heterocyclyl” group refers to all heterocyclyl groups with 4 to 6 total ring atoms, for example, azetidine, oxetane, oxazoline, pyrrolidine, piperidine, piperazine, morpholine, and the like. If no “a” and “b” are designated with regard to an alkyl, alkenyl, alkynyl, cycloalkyl, or aryl group, the broadest range described in these definitions is to be assumed. As used herein, the term “C₁-C₆” includes C₁, C₂, C₃, C₄, C₅ and C₆, and a range defined by any of the two numbers. For example, C₁-C₆ alkyl includes C₁, C₂, C₃, C₄, C₅ and C₆ alkyl, C₂-C₆ alkyl, C₁-C₃ alkyl, etc. Similarly, C₂-C₆ alkenyl includes C₂, C₃, C₄, C₅ and C₆ alkenyl, C₂-C₅ alkenyl, C₃-C₄ alkenyl, etc.; and C₂-C₆ alkynyl includes C₂, C₃, C₄, C₅ and C₆ alkynyl, C₂-C₅ alkynyl, C₃-C₄ alkynyl, etc. C₃-C₈ cycloalkyl each includes hydrocarbon ring containing 3, 4, 5, 6, 7 and 8 carbon atoms, or a range defined by any of the two numbers, such as C₃-C₇ cycloalkyl or C₅-C₆ cycloalkyl.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 6 carbon atoms. The alkyl group may be designated as “C₁-C₄ alkyl” or similar designations. By way of example only, “C₁-C₆ alkyl” indicates that there are one to six carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkoxy” refers to the formula —OR wherein R is an alkyl as is defined above, such as “C₁-C₉ alkoxy,” including but not limited to methoxy, ethoxy, n-propoxy, 1-methylethoxy (isopropoxy), n-butoxy, iso-butoxy, sec-butoxy, and tert-butoxy, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 6 carbon atoms. The alkenyl group may be designated as “C₂-C₆ alkenyl” or similar designations. By way of example only, “C₂-C₆ alkenyl” indicates that there are two to six carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆-C₁₀ aryl,” “C₆ or C₁₀ aryl,” or similar designations. Examples of aryl groups include, but are not limited to, phenyl, naphthyl, azulenyl, and anthracenyl.

An “aralkyl” or “arylalkyl” is an aryl group connected, as a substituent, via an alkylene group, such as “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁-C₆ alkylene group).

As used herein, “aryloxy” refers to RO— in which R is an aryl, as defined above, such as but not limited to phenyl.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinolinyl, benzoimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁-C₆ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃-C₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

As used herein, “cycloalkyl” means a fully saturated carbocyclyl ring or ring system. Examples include cyclopropyl, cyclobutyl, cyclopentyl, and cyclohexyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

As used herein, “(aryl)alkyl” refer to an aryl group, as defined above, connected, as a substituent, via an alkylene group, as described above. The alkylene and aryl group of an aralkyl may be substituted or unsubstituted. Examples include but are not limited to benzyl, 2-phenylalkyl, 3-phenylalkyl, and naphthylalkyl. In some embodiments, the alkylene is an unsubstituted straight chain containing 1, 2, 3, 4, 5, or 6 methylene unit(s).

As used herein, “(heteroaryl)alkyl” refer to a heteroaryl group, as defined above, connected, as a substituent, via an alkylene group, as defined above. The alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl, and imidazolylalkyl, and their benzo-fused analogs. In some embodiments, the alkylene is an unsubstituted straight chain containing 1, 2, 3, 4, 5, or 6 methylene unit(s).

As used herein, “(heterocyclyl)alkyl” refer to a heterocyclic or a heterocyclyl group, as defined above, connected, as a substituent, via an alkylene group, as defined above. The alkylene and heterocyclyl groups of a (heterocyclyl)alkyl may be substituted or unsubstituted. Examples include but are not limited to (tetrahydro-2H-pyran-4-yl)methyl, (piperidin-4-yl)ethyl, (piperidin-4-yl) propyl, (tetrahydro-2H-thiopyran-4-yl)methyl, and (1,3-thiazinan-4-yl)methyl. In some embodiments, the alkylene is an unsubstituted straight chain containing 1, 2, 3, 4, 5, or 6 methylene unit(s).

As used herein, “(carbocyclyl)alkyl” refer to a carbocyclyl group (as defined herein) connected, as a substituent, via an alkylene group. Examples include but are not limited to cyclopropylmethyl, cyclobutylmethyl, cyclopentylethyl, and cyclohexylpropyl. In some embodiments, the alkylene is an unsubstituted straight chain containing 1, 2, 3, 4, 5, or 6 methylene unit(s).

As used herein, “alkoxyalkyl” or “(alkoxy)alkyl” refers to an alkoxy group connected via an alkylene group, such as C₂-C₈ alkoxyalkyl, or (C₁-C₆ alkoxy) C₁-C₆ alkyl, for example, —(CH₂)₁₋₃—OCH₃.

As used herein, “—O-alkoxyalkyl” or “—O-(alkoxy)alkyl” refers to an alkoxy group connected via an —O-(alkylene) group, such as —O—(C₁-C₆ alkoxy) C₁-C₆ alkyl, for example, —O—(CH₂)₁₋₃—OCH₃.

As used herein, “haloalkyl” refers to an alkyl group in which one or more of the hydrogen atoms are replaced by a halogen (e.g., mono-haloalkyl, di-haloalkyl, and tri-haloalkyl). Such groups include but are not limited to, chloromethyl, fluoromethyl, difluoromethyl, trifluoromethyl and 1-chloro-2-fluoromethyl, 2-fluoroisobutyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy and 1-chloro-2-fluoromethoxy, 2-fluoroisobutoxy. A haloalkoxy may be substituted or unsubstituted.

An “amino” group refers to a —NH₂ group. The term “mono-substituted amino group” as used herein refers to an amino (—NH₂) group where one of the hydrogen atom is replaced by a substituent. The term “di-substituted amino group” as used herein refers to an amino (—NH₂) group where each of the two hydrogen atoms is replaced by a substituent. The term “optionally substituted amino,” as used herein refer to a —NR_{A}R_{B} group where R_{A} and R_{B} are independently hydrogen, alkyl, cycloalkyl, aryl, heteroaryl, heterocyclyl, aralkyl, or heterocyclyl(alkyl), as defined herein.

An “O-carboxy” group refers to a “—OC(═O)R” group in which R is selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “C-carboxy” group refers to a “—C(O)OR” group in which R is selected from the group consisting of hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein. A non-limiting example includes carboxyl (i.e., —C(═O)OH).

A “sulfonyl” group refers to an “—SO₂R” group in which R is selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “sulfino” group refers to a “—S(═O)OH” group.

A “sulfo” group refers to a “—S(═O)₂OH” or “—SO₃H” group.

A “sulfonate” group refers to a “—SO₃⁻” group.

A “sulfate” group refers to “—SO₄⁻” group.

A “S-sulfonamido” group refers to a “—SO₂NR_{A}R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “N-sulfonamido” group refers to a “—N(R_{A}) SO₂R_{B}” group in which R_{A} and R_{b} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “C-amido” group refers to a “—C(═O)NR_{A}R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “N-amido” group refers to a “—N(R_{A})C(═O)R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “O-carbamyl” group refers to a “—OC(═O)N(R_{A}R_{B})” group in which R_{A} and R_{B} can be the same as defined with respect to S-sulfonamido. An O-carbamyl may be substituted or unsubstituted.

An “N-carbamyl” group refers to an “ROC(═O)N(R_{A})—” group in which R and R_{A} can be the same as defined with respect to N-sulfonamido. An N-carbamyl may be substituted or unsubstituted.

An “O-thiocarbamyl” group refers to a “—OC(═S)—N(R_{A}R_{B})” group in which R_{A} and R_{B} can be the same as defined with respect to S-sulfonamido. An O-thiocarbamyl may be substituted or unsubstituted.

An “N-thiocarbamyl” group refers to an “ROC(═S)N(R_{A})—” group in which R and R_{A} can be the same as defined with respect to N-sulfonamido. An N-thiocarbamyl may be substituted or unsubstituted.

The term “alkylamino” or “(alkyl)amino” refers to an amino group wherein one or both hydrogen is replaced by an alkyl group.

An “(alkoxy)alkyl” group refers to an alkoxy group connected via an alkylene group, such as a “(C₁-C₆ alkoxy) C₁-C₆ alkyl” and the like.

The term “hydroxy” as used herein refers to a “—OH” group.

The term “cyano” group as used herein refers to a “—CN” group.

The term “azido” as used herein refers to a “—N₃” group.

The term “isonitrile” as used herein refers to a “—N⁺≡C⁻” group.

When a group is described as “optionally substituted” it may be either unsubstituted or substituted. Likewise, when a group is described as being “substituted,” the substituent may be selected from one or more of the indicated substituents. As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇ carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), (aryl) C₁-C₆ alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), (5-10 membered heteroaryl)C₁-C₆ alkyl (optionally substituted with halo, C₁-C₆ alkyl, C₁-C₆ alkoxy, C₁-C₆ haloalkyl, and C₁-C₆ haloalkoxy), halo, —CN, hydroxy, C₁-C₆ alkoxy, (C₁-C₆ alkoxy) C₁-C₆ alkyl, —O(C₁-C₆ alkoxy)C₁-C₆ alkyl; (C₁-C₆ haloalkoxy) C₁-C₆ alkyl; —O(C₁-C₆ haloalkoxy)C₁-C₆ alkyl; aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino (C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, —SO₃H, sulfonate, sulfate, sulfino, —OSO₂C₁₋₄alkyl, monophosphate, diphosphate, triphosphate, and oxo (═O). Wherever a group is described as “optionally substituted” that group can be substituted with the above substituents.

In each instance where a single mesomeric form of a compound described herein is shown, the alternative mesomeric forms are equally contemplated.

As used herein, a “nucleotide” includes a nitrogen containing heterocyclic base, a sugar, and one or more phosphate groups. They are monomeric units of a nucleic acid sequence. In RNA, the sugar is a ribose, and in DNA a deoxyribose, i.e., a sugar lacking a hydroxyl group that is present in ribose. The nitrogen containing heterocyclic base can be purine, deazapurine, or pyrimidine base. Purine bases include adenine (A) and guanine (G), and modified derivatives or analogs thereof, such as 7-deaza adenine or 7-deaza guanine. Pyrimidine bases include cytosine (C), thymine (T), and uracil (U), and modified derivatives or analogs thereof. The C-1 atom of deoxyribose is bonded to N-1 of a pyrimidine or N-9 of a purine.

As used herein, a “nucleotide conjugate” generally refers to a nucleotide labeled with a fluorescent moiety, optionally through a cleavage linker as described herein. In some embodiment, when a nucleotide conjugate is described as an unlabeled nucleotide, such nucleotide does not include a fluorescent moiety. In some further embodiments, an unlabeled nucleotide conjugate also does not have a cleavable linker.

As used herein, a “nucleoside” is structurally similar to a nucleotide but is missing the phosphate moieties. An example of a nucleoside analogue would be one in which the label is linked to the base and there is no phosphate group attached to the sugar molecule. The term “nucleoside” is used herein in its ordinary sense as understood by those skilled in the art. Examples include, but are not limited to, a ribonucleoside comprising a ribose moiety and a deoxyribonucleoside comprising a deoxyribose moiety. A modified pentose moiety is a pentose moiety in which an oxygen atom has been replaced with a carbon and/or a carbon has been replaced with a sulfur or an oxygen atom. A “nucleoside” is a monomer that can have a substituted base and/or sugar moiety. Additionally, a nucleoside can be incorporated into larger DNA and/or RNA polymers and oligomers.

The term “purine base” is used herein in its ordinary sense as understood by those skilled in the art and includes its tautomers. Similarly, the term “pyrimidine base” is used herein in its ordinary sense as understood by those skilled in the art and includes its tautomers. A non-limiting list of optionally substituted purine-bases includes purine, adenine, guanine, deazapurine, 7-deaza adenine, 7-deaza guanine. hypoxanthine, xanthine, alloxanthine, 7-alkylguanine (e.g., 7-methylguanine), theobromine, caffeine, uric acid and isoguanine. Examples of pyrimidine bases include, but are not limited to, cytosine, thymine, uracil, 5,6-dihydrouracil and 5-alkylcytosine (e.g., 5-methylcytosine).

As used herein, when an oligonucleotide or polynucleotide is described as “comprising” a nucleoside or nucleotide described herein, it means that the nucleoside or nucleotide described herein forms a covalent bond with the oligonucleotide or polynucleotide. Similarly, when a nucleoside or nucleotide is described as part of an oligonucleotide or polynucleotide, such as “incorporated into” an oligonucleotide or polynucleotide, it means that the nucleoside or nucleotide described herein forms a covalent bond with the oligonucleotide or polynucleotide. In some such embodiments, the covalent bond is formed between a 3′ hydroxy group of the oligonucleotide or polynucleotide with the 5′ phosphate group of a nucleotide described herein as a phosphodiester bond between the 3′ carbon atom of the oligonucleotide or polynucleotide and the 5′ carbon atom of the nucleotide.

As used herein, the term “cleavable linker” is not meant to imply that the whole linker is required to be removed. The cleavage site can be located at a position on the linker that ensures that part of the linker remains attached to the detectable label and/or nucleoside or nucleotide moiety after cleavage.

As used herein, “derivative” or “analog” means a synthetic nucleotide or nucleoside derivative having modified base moieties and/or modified sugar moieties. Such derivatives and analogs are discussed in, e.g., Scheit, Nucleotide Analogs (John Wiley & Son, 1980) and Uhlman et al., Chemical Reviews 90:543-584, 1990. Nucleotide analogs can also comprise modified phosphodiester linkages, including phosphorothioate, phosphorodithioate, alkyl-phosphonate, and phosphoramidate linkages. “Derivative,” “analog” and “modified” as used herein, may be used interchangeably, and are encompassed by the terms “nucleotide” and “nucleoside” defined herein.

As used herein, the term “phosphate” is used in its ordinary sense as understood by those skilled in the art, and includes its protonated forms (for example,

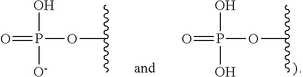 [SMILES: *OP(=O)([O-])O ; *OP(=O)(O)O]

As used herein, the terms “monophosphate,” “diphosphate,” and “triphosphate” are used in their ordinary sense as understood by those skilled in the art and include protonated forms.

As understood by one of ordinary skill in the art, a compound such as a nucleotide conjugate described herein may exist in ionized form, e.g., containing a —CO₂⁻, —SO₃⁻ or —O⁻. If a compound contains a positively or negatively charged substituent group, it may also contain a negatively or positively charged counterion such that the compound as a whole is neutral. In other aspects, the compound may exist in a salt form, where the counterion is provided by a conjugate acid or base.

As used herein, the term “phasing” refers to a phenomenon in SBS that is caused by incomplete removal of the 3′ terminators and fluorophores, and/or failure to complete the incorporation of a portion of DNA strands within clusters by polymerases at a given sequencing cycle. Prephasing is caused by the incorporation of nucleotides without effective 3′ terminators, wherein the incorporation event goes 1 cycle ahead due to a termination failure. Phasing and prephasing cause the measured signal intensities for a specific cycle to consist of the signal from the current cycle as well as noise from the preceding and following cycles. As the number of cycles increases, the fraction of sequences per cluster affected by phasing and prephasing increases, hampering the identification of the correct base. Prephasing can be caused by the presence of a trace amount of unprotected or unblocked 3′-OH nucleotides during sequencing by synthesis (SBS). The unprotected 3′-OH nucleotides could be generated during the manufacturing processes or possibly during the storage and reagent handling processes.

### Conjugated Polymers/Polymer Dots

One aspect of the present disclosure relates to conjugated polymers or polymer dots for use as detectable labels, as alternatives to small molecule organic dyes. In some aspect, a water-soluble conjugated polymer or water-dispersible polymer dot in accordance with the present disclosure can bind to a nucleotide, oligonucleotide or polynucleotide and may act as a detectable label of the nucleotide, oligonucleotide or polynucleotide. In some examples, the water-soluble conjugated polymer or polymer dot bind to the nucleotide, oligonucleotide or polynucleotide via covalent bonding. For example, a reactive moiety of the nucleotide, oligonucleotide or polynucleotide forms covalent bond with a functional moiety of the water-soluble conjugated polymer or water-dispersible polymer dot via a biorthogonal reaction selected from the group consisting of a [3+2] dipolar cycloaddition, a Diels-Alder cycloaddition, a [4+1] cycloaddition, a phosphine ligation, or condensation with 2-acylphenyl boronic acid. In some other examples, the water-soluble conjugated polymer or polymer dot bind the nucleotide, oligonucleotide or polynucleotide via non-covalent interactions.

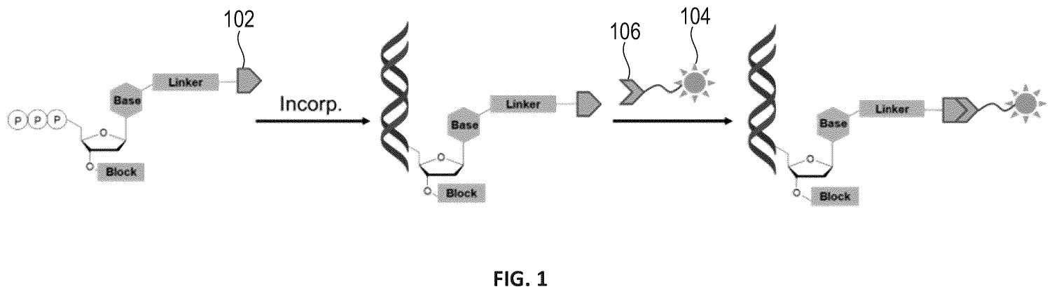

FIG. 1 schematically illustrates an exemplary SBS scheme where a conjugated polymer or polymer dot labels an incorporated nucleotide. A nucleotide having a reactive moiety 102 is first incorporated into to a DNA strand in the presence of a DNA polymerase. Then, a conjugated polymer or polymer dot 104 having a functional moiety 106 is introduced, and the reactive moiety 102 binds to the functional moiety 106.

### Water-Soluble Conjugated Polymers

In some embodiments, the water-soluble conjugated polymer comprises one or more repeating unit moieties selected from the group consisting of:

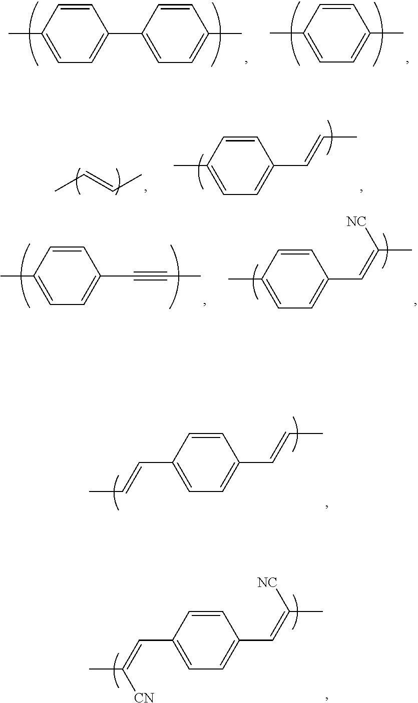

a five membered heteroarylene containing one, two or three heteroatoms selected from the group consisting of N, O and S,

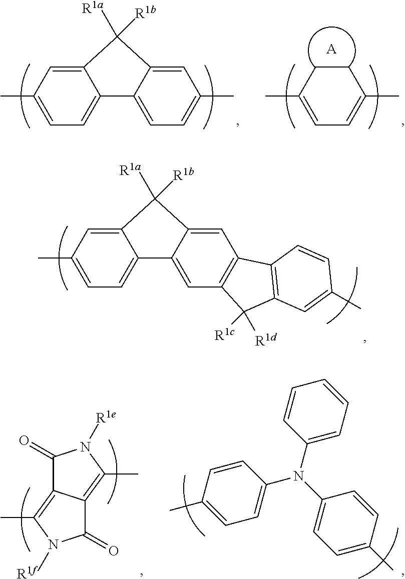

and combinations thereof;
wherein each of R^{1a}, R^{1b}, R^{1c}, R^{1d}, R^{1e} and R^{1f} is independently H, unsubstituted C₁-C₁₂ alkyl, C₁-C₁₂ alkyl substituted with one or more water solubilizing groups, or 2 to 20 membered heteroalkyl;
ring A is a five or six membered heteroaryl containing one, two or three heteroatoms selected from the group consisting of N, O and S, and
wherein each structure is optionally substituted with one or more electron donating groups, one or more electron withdrawing groups, one or more functional moiety R_{F}, or one or more water solubilizing group R^{S}, or combinations thereof.

In some embodiments of the water-soluble conjugated polymer described herein, the electron donating groups comprise a C₁-C₁₂ alkoxy, for example —OC₈H₁₇. In some embodiments, electron withdrawing group includes —CN, nitro, fluoro, or trifluoromethyl. In some further embodiments, the water solubilizing groups are selected from the group consisting of phosphate;

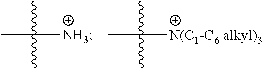

(wherein each C₁-C₆ alkyl in

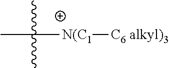

is optionally substituted with one or more substituents selected from phosphate,

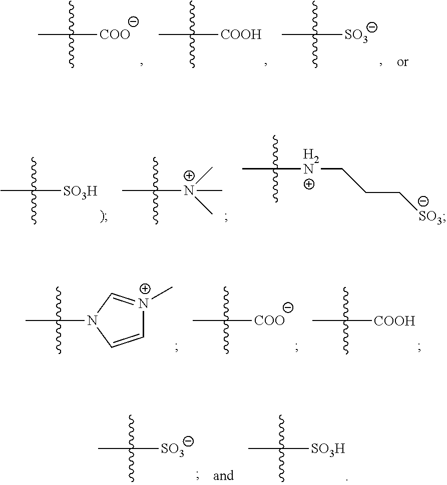

Alternative water solubilizing group include ethylene glycol repeating units (—CH₂CH₂O)— which may be incorporated into an alkyl group described herein. In some further embodiments, the conjugated polymer is covalently bounded to the nucleotide, oligonucleotide or polynucleotide through reaction of a functional moiety R^{F} of the conjugated polymer with a reactive moiety of the oligonucleotide or polynucleotide. In yet a further embodiment, the conjugated polymer comprises the structure:

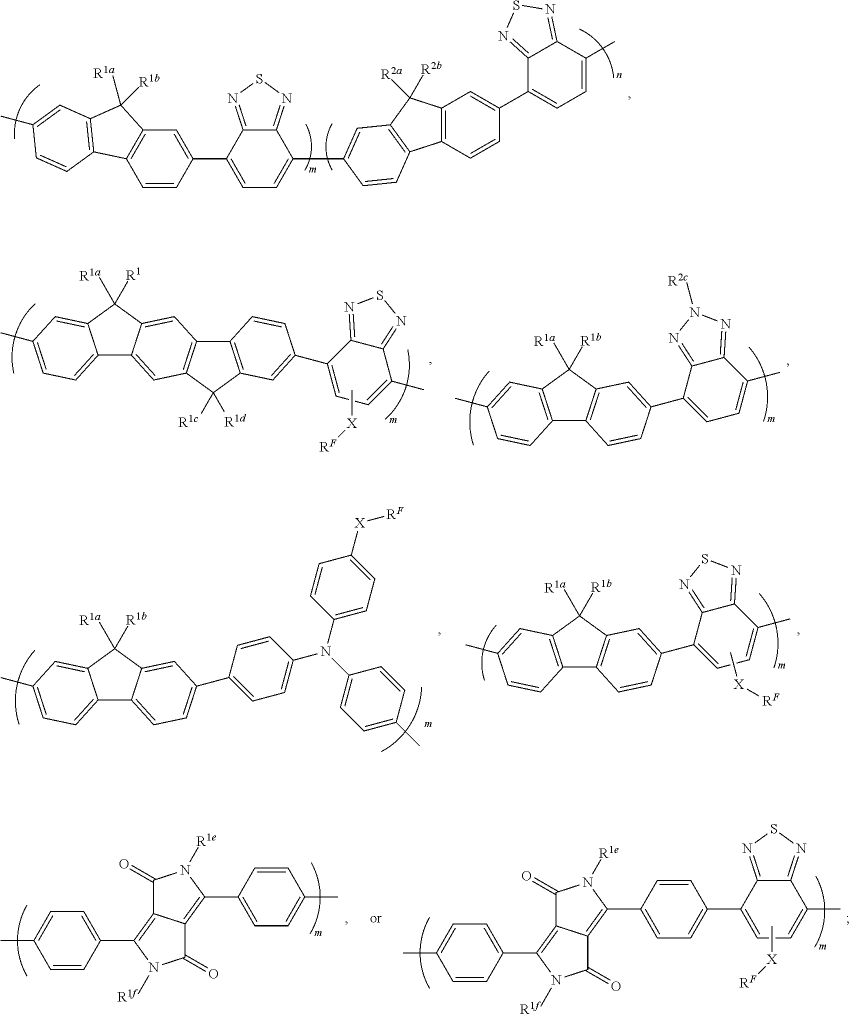

wherein each of R^{2a}, R^{2b} and R^{2c} is independently H, R^{F} or C₁-C₁₂ alkyl substituted with R^{F};
X is O, S, an alkylene linker, or a heteroalkylene linker (such as polyethylene glycol with 2 to 100 repeating units (—OCH₂CH₂));
R^{F} is azido, amino, a reactive ester,

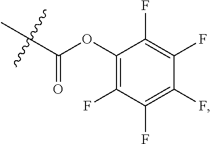 [SMILES: *C(=O)Oc1c(F)c(F)c(F)c(F)c1F]

unsubstituted or substituted 1,2,4,5-tetrazinyl,

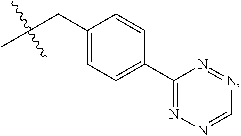 [SMILES: *Cc1ccc(-c2nncnn2)cc1]

unsubstituted or substituted dibenzocyclooctyne (DBCO),

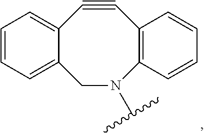 [SMILES: *N1Cc2ccccc2C#Cc2ccccc21]

unsubstituted or substituted bicyclo[6.1.0]nonynyl (BCN),

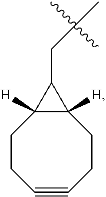

transcyclooctenyl (TCO), methylcyclooctene

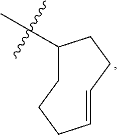 [SMILES: *C1CC/C=C/CCC1]

and each of m and n is independently an integer from 1 to 100,000. In some embodiments, each of R^{1a}, R^{1b}, R^{1c}, R^{1d}, R^{1e} and R^{1f} is —C₈H₁₇, optionally substituted with one or more water solubilizing groups selected from the group consisting of phosphate;

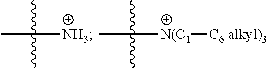

(wherein each C₁-C₆ alkyl in

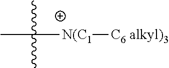

is optionally substituted with one or more substituents selected from phosphate,

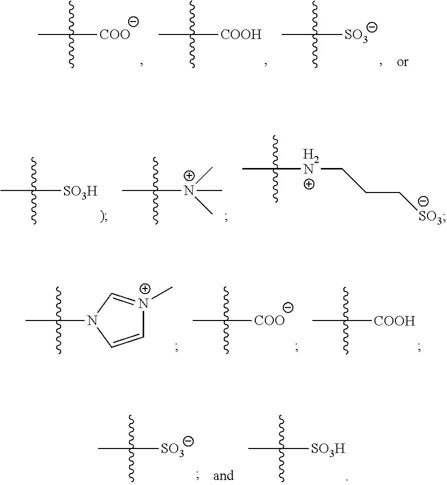

In other embodiments, each of R^{1a}, R^{1b}, R^{1c}, R^{1d}, R^{1e} and R^{1f} is independently 2-20 membered heteroalkyl comprising 2, 3, 4, 5, or 6 polyethylene glycol unit (—OCH₂CH₂).

In some embodiments, the conjugated polymer is attached covalently to a nucleotide, oligonucleotide, or polynucleotide via covalent bonding via the R^{F} group as described herein. In some embodiments, the conjugated polymer is attached to the nucleotide, oligonucleotide, or polynucleotide via non-covalent bonding. For example, the R^{F} group contains a biotin moiety

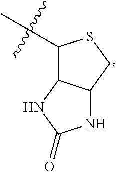 [SMILES: *C1SCC2NC(=O)NC21]

which can form non-covalent bonding with an avidin moiety (such as streptavidin), or vice versa.

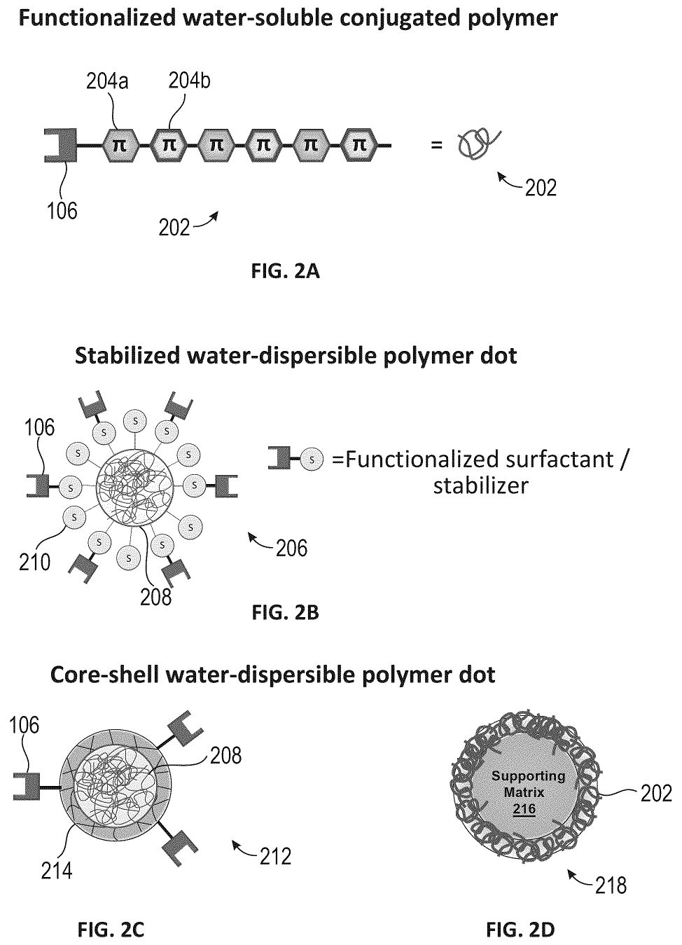

FIG. 2A is a cartoon drawing of a functionalized water-soluble conjugated polymer 202. The functionalized water-soluble conjugated polymer 202 includes a functional moiety 106. The functionalized water-soluble conjugated polymer 202 can include repeating units, for example units 204a and 204b. The units 204a and/or 204b may be capable of emitting a detectable signal in response to excitation. For example, the units 204a and/or 204b can emit fluorescence in response to photo excitation.

### Water-Dispersible Polymer Dots Stabilized by Functionalized Surfactants

In some embodiments, the polymer dot is in the form of a water-dispersible polymer dot comprising a conjugated polymer nanoparticle and one or more functionalized surfactants attached to the surface of the nanoparticle through non-covalent interaction. In some further embodiments, the conjugated polymer comprises one or more repeating unit moieties having a structure selected from the group consisting of

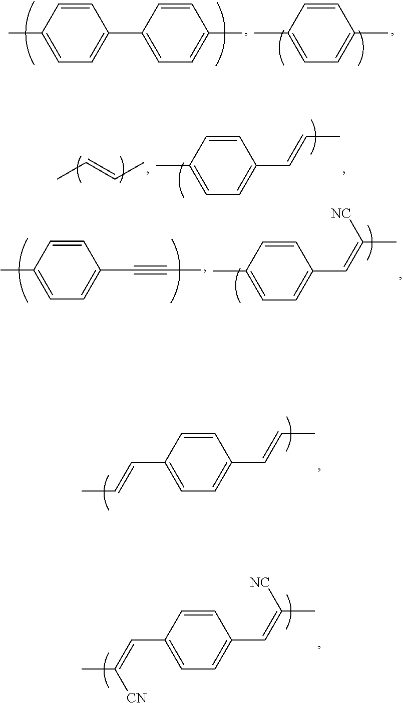

a five membered heteroarylene containing one, two or three heteroatoms selected from the group consisting of N, O and S,

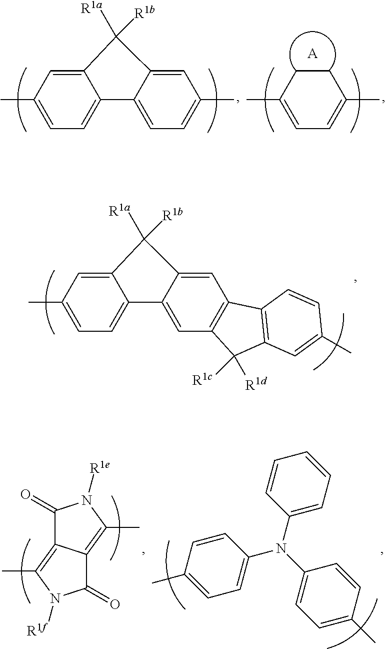

and combinations thereof;
wherein each of R^{1a}, R^{1b}, R^{1c}, R^{1d}, R^{1e} and R^{1f} is independently H, unsubstituted C₁-C₁₂ alkyl, C₁-C₁₂ alkyl substituted with one or more water solubilizing groups, or 2 to 20 membered heteroalkyl;
ring A is a five or six membered heteroaryl containing one, two or three heteroatoms selected from the group consisting of N, O and S, and
wherein each structure is optionally substituted with one or more electron donating groups, one or more electron withdrawing groups, one or more functional moiety R^{F}, or one or more water solubilizing group R^{S}, or combinations thereof. In some embodiments, the electron donating groups comprise a C₁-C₁₂ alkoxy, for example, —OC₈H₁₇. In some embodiments, electron withdrawing group includes —CN, nitro, fluoro, or trifluoromethyl. In some further embodiments, R^{S} is selected from the group consisting of phosphate;

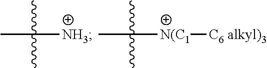

(wherein each C₁-C₆ alkyl is optionally substituted with one or more substituents selected from phosphate,

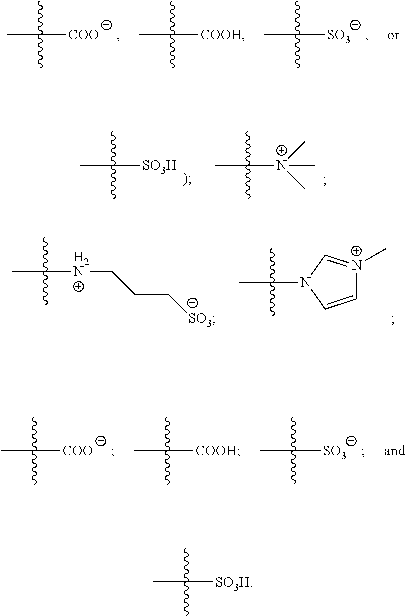

In yet further embodiments, the conjugated polymer comprises:

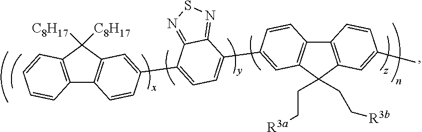

poly(9,9-dioctylfluorene-alt-benzothiadiazole) (PFBT,

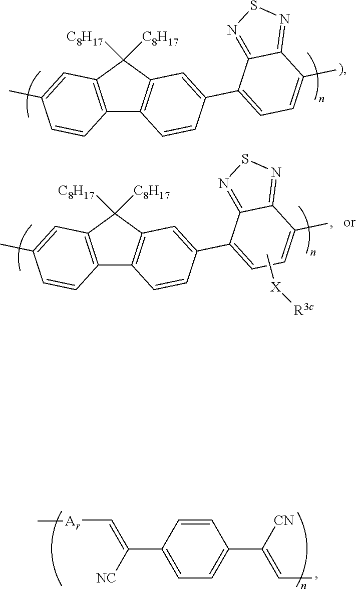

wherein each of R^{3a}, R^{3b} and R^{3c} is independently R^{S} or R^{F};
Aᵣ is

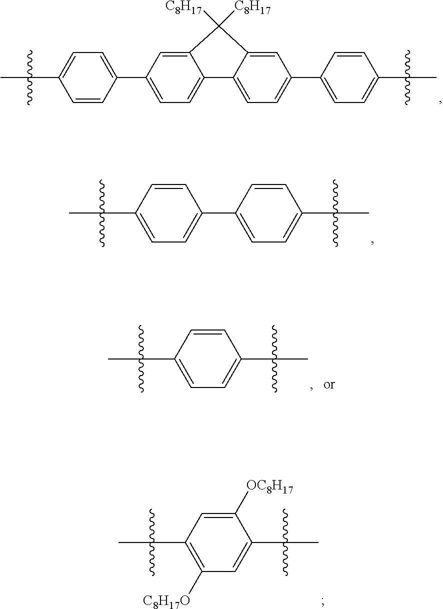

X is O, S, an alkylene linker, or a heteroalkylene linker (such as polyethylene glycol with 2 to 100 repeating units (—OCH₂CH₂));
R^{F} is azido, amino, a reactive ester,

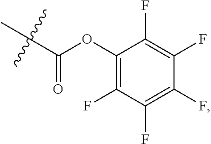 [SMILES: *C(=O)Oc1c(F)c(F)c(F)c(F)c1F]

unsubstituted or substituted 1,2,4,5-tetrazinyl,

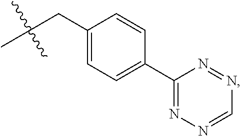 [SMILES: *Cc1ccc(-c2nncnn2)cc1]

unsubstituted or substituted dibenzocyclooctyne (DBCO),

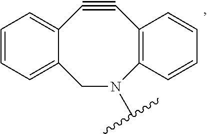 [SMILES: *N1Cc2ccccc2C#Cc2ccccc21]

unsubstituted or substituted bicyclo[6.1.0]nonynyl (BCN),

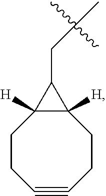

transcyclooctenyl (TCO), or

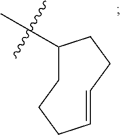 [SMILES: *C1CC/C=C/CCC1]

and
each of m, n, x, y and z is independently an integer from 1 to 100,000.

In some embodiments, the water-dispersible polymer dot is covalently bounded to the nucleotide, oligonucleotide or polynucleotide through reaction of a functional moiety of the conjugated polymer or a functional moiety of the one or more functionalized surfactants with a reactive moiety of the nucleotide, oligonucleotide or polynucleotide. In some further embodiments, the functional moiety of the conjugated polymer or one or more functionalized surfactants is selected from the group consisting of azido, amino, a reactive ester,

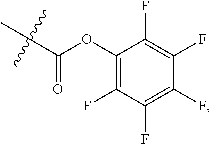 [SMILES: *C(=O)Oc1c(F)c(F)c(F)c(F)c1F]

unsubstituted or substituted 1,2,4,5-tetrazinyl,

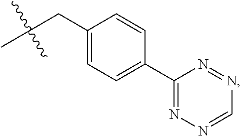 [SMILES: *Cc1ccc(-c2nncnn2)cc1]

unsubstituted or substituted DBCO,

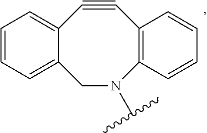 [SMILES: *N1Cc2ccccc2C#Cc2ccccc21]

unsubstituted or substituted BCN

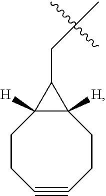

and unsubstituted or substituted TCO

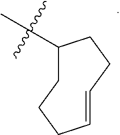 [SMILES: *C1CC/C=C/CCC1]

In some other embodiments, the water-dispersible polymer dot is attached to a nucleotide, oligonucleotide, or polynucleotide via non-covalent interactions. For example, a functional moiety

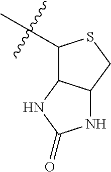 [SMILES: *C1SCC2NC(=O)NC21]

of the conjugated polymer or the functionalized surfactants forms non-covalent bonding with an avidin (e.g., streptavidin) moiety of the nucleotide, oligonucleotide or polynucleotide, or vice versa.

In some further embodiments of the water-dispersible polymer dot as described herein, the one or more functionalized surfactants comprises an amphiphilic block copolymer or a water-soluble nonionic emulsifier (e.g., Polysorbate 80 (Tween® 80)), or a combination thereof. In some further embodiments, the amphiphilic block copolymer comprises poly(styrene)-block-poly(ethylene glycol) acetic acid (PS-PEG-COOH)

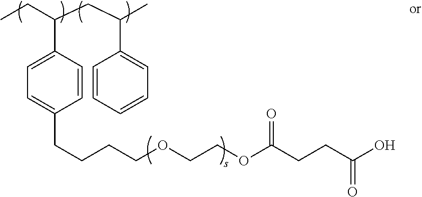

poly(styrene)-block-poly(ethylene glycol) azide (PS-PEG-N₃)

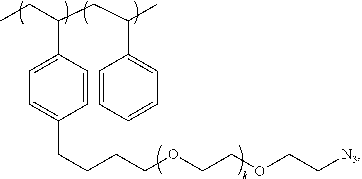

each of s and k is independently an integer from 1 to 10,000.

In other embodiments, the polymer dot can be self-stabilized by a functionalized polar side chain on the conjugated polymer and there is no surfactant added.

### Core-Shell Water-Dispersible Polymer Dots

In some other embodiments, the polymer dot described herein is in the form of a core-shell water-dispersible polymer dot, wherein the core-shell water-dispersible polymer dot comprises a conjugated polymer nanoparticle core as described herein, and a hydrogel shell. In some such embodiments, the core-shell water-dispersible polymer dot further comprises one or more surfactants on the surface of the conjugated polymer nanoparticle core. In some further embodiments, the one or more surfactants comprises a water soluble nonionic emulsifier (e.g., Polysorbate 80 (Tween® 80)) or an anionic surfactant (e.g., sodium dodecyl sulfate (SDS)), or a combination thereof. In some further embodiments, the core-shell water-dispersible polymer dot is covalently bounded to the oligonucleotide or polynucleotide through reaction of a functional moiety of the hydrogel shell with a reactive moiety of the nucleotide, oligonucleotide or polynucleotide.

In some other embodiments, the polymer dot described herein is in the form of a core-shell water-dispersible polymer dot comprises a water-soluble conjugated polymer as described herein, coated on a supporting particle matrix core. In some such embodiments, the core-shell water-dispersible polymer dot is covalently bounded to the nucleotide, oligonucleotide or polynucleotide through reaction of a functional moiety of the water-soluble conjugated polymer or the supporting particle matrix with a reactive moiety of the nucleotide, oligonucleotide or polynucleotide. In some embodiments, the supporting particle matrix comprises a water-soluble polymer (e.g., polyacrylamide), micelles or silica. In other embodiments, the conjugated polymer is a hydrophilic, water-swellable polymer.

In some embodiments, the functional moiety of the hydrogel shell, the functional moiety of the water-soluble conjugated polymer or the functional moiety of the supporting particle matrix is selected from the group consisting of azido, amino, a reactive ester,

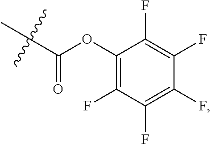 [SMILES: *C(=O)Oc1c(F)c(F)c(F)c(F)c1F]

unsubstituted or substituted 1,2,4,5-tetrazinyl,

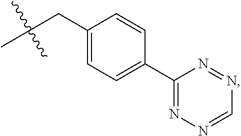 [SMILES: *Cc1ccc(-c2nncnn2)cc1]

unsubstituted or substituted DBCO,

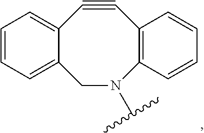 [SMILES: *N1Cc2ccccc2C#Cc2ccccc21]

unsubstituted or substituted BCN,

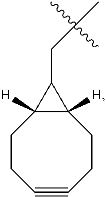

unsubstituted or substituted TCO, and

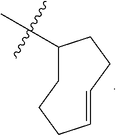 [SMILES: *C1CC/C=C/CCC1]

In some further embodiments, the hydrogel shell comprises an acrylamide hydrogel. In some such embodiments, the acrylamide hydrogel is a copolymer of N-isopropylacrylamide (NiPAM), bisacrylamide (bisAM), and acrylic acid (AAc).

In some embodiments of the functional/reactive moiety described herein, an azido may include primary azide such as

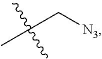 [SMILES: *CN=[N+]=[N-]]

aryl azide such as

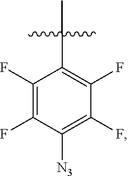 [SMILES: *c1c(F)c(F)c(N=[N+]=[N-])c(F)c1F]

or tertiary azide such as

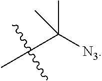 [SMILES: *C(C)(C)N=[N+]=[N-]]

1,2,4,5-tetrazine may include phenyl tetrazine

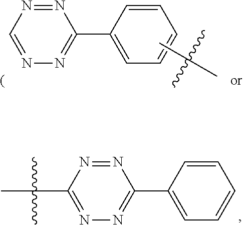

wherein the phenyl ring may be optionally substituted), pyrimidyl tetrazine

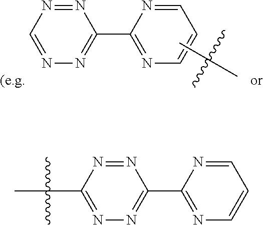

wherein the pyrimidyl ring is optionally substituted), methyl tetrazine

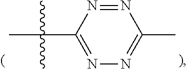 [SMILES: *c1nnc(C)nn1]

pyridyl tetrazine

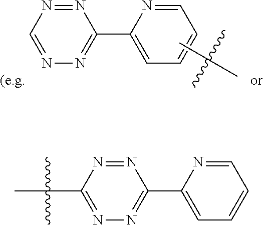

where the pyridyl ring is optionally substituted), t-butyl tetrazine

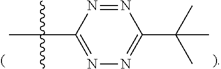 [SMILES: *c1nnc(C(C)(C)C)nn1]

In some embodiments, the polymer dot described herein (including both water-dispersible polymer dot stabilized with functionalized surfactant and core-shell water-dispersible polymer dot) has a diameter of from about 1 nm to about 1000 nm, about 2 nm to about 500 nm, about 5 nm to 100 nm, about 10 nm to about 50 nm, about 1 nm to about 50 nm, about 2 nm and about 40 nm, about 4 nm to about 30 nm, or about 5 nm to about 20 nm, or about 10 nm. In some embodiments, the ratio between the size of the core and the size of the shell can be anything between 99% to 1% core size to shell size to about 10% to 90% core size to shell size, for example between about 95% to 5%, 90% to 10%, 80% to 20%, 70% to 30%, 60% to 40%, 50% to 50%, 40% to 60%, 30% to 70%, 20% to 80% or 10% to 90% core-shell size ratio.

FIG. 2B is a cartoon drawing of a stabilized water-dispersible polymer dot 206. The stabilized water-dispersible polymer dot 206 can include functional moieties 106, a polymer dot core 208, and surfactants 210. The stabilized water-dispersible polymer dot 206 is capable of emitting a detectable signal in response to excitation, for example, in response to photo excitation. One or more of the surfactants 210 may be functionalized by binding to a functional moiety 106. The surfactants 210 may stabilize the polymer dot core 208 by binding to an outer surface of the polymer dot core 208.

FIG. 2C is a cartoon drawing of a core-shell water-dispersible polymer dot 212. The core-shell water-dispersible polymer dot 212 can include functional moieties 106, a polymer core 208, and a hydrogel shell 214. The polymer dot 212 is capable of emitting a detectable signal in response to excitation, for example, in response to photoexcitation. The hydrogel shell 214 may be functionalized by binding to one or more functional moieties 106. The hydrogel shell 214 may stabilize the polymer core 208 by binding to an outer surface of the polymer core 208.

FIG. 2D is a cartoon drawing of another core-shell water-dispersible polymer dot 218. The core-shell water-dispersible polymer dot 218 includes a supporting matrix core 216, which acts as a core of the core-shell water-dispersible polymer dot 218. A functionalized water-soluble conjugated polymer 202 makes up the shell of the core-shell water-dispersible polymer dot 218.

### Synthesis of Conjugated Polymers and Polymer Dots

As described herein, water dispersible polymer dots (conjugated polymer particles) stabilized by functionalized surfactants may be prepared from two different methods. A first method is precipitation and encapsulation method from pre-formed conjugated polymers. In this method, conjugated polymers with the desired optoelectronic properties are synthesized in solution and the polymer dots are generated post-synthesis by precipitation methods. Functionality on the polymer dot can be introduced either through: i) non-covalent interactions by using a surfactant or co-precipitation with a amphiphilic polymer with functional groups (e.g. PS-PEG-COOH, PS-PEG-N₃, or styrene-maleic anhydride copolymers (PSMA)); ii) direct functionalization of the conjugated polymer side chain or backbone. A second method is in-situ emulsion polymerization using functionalized surfactants. In this method, polymer dots are synthesized via in-situ emulsion polymerization using Knoevenagel condensation reaction, aldol condensation, and/or Suzuki-Miyaura cross-coupling. Then, a functionalized co-surfactant (telechelic polystyrene-based amphiphilic block copolymers as described herein) is introduced to the surface of the polymer dots. Similar methods were previously reported in J. Am. Chem. Soc. 2010, 132, 15410-15417, ACS Nano 2012, 6, 5429-5439, Nat. Comm. 2018, 9, 3237 and Chem. Commun. 2010, 46, 1617-1619.

As described herein, core-shell water dispersible polymer dots can also be prepared by two different methods. In a first method, polymer dots are synthesized via in-situ emulsion polymerization using Knoevenagel condensation (or aldol condensation, and/or Suzuki-Miyaura cross-coupling) with a co-surfactant (e.g., sodium dodecyl sulfate (SDS)). Then, forming a hydrogel (e.g., acrylamide based hydrogel) shell outside the polymer dot cores. In a second inverted method, the core-shell structure can also include coating the water-soluble conjugated polymers on any supporting particle matrix, including those described herein.

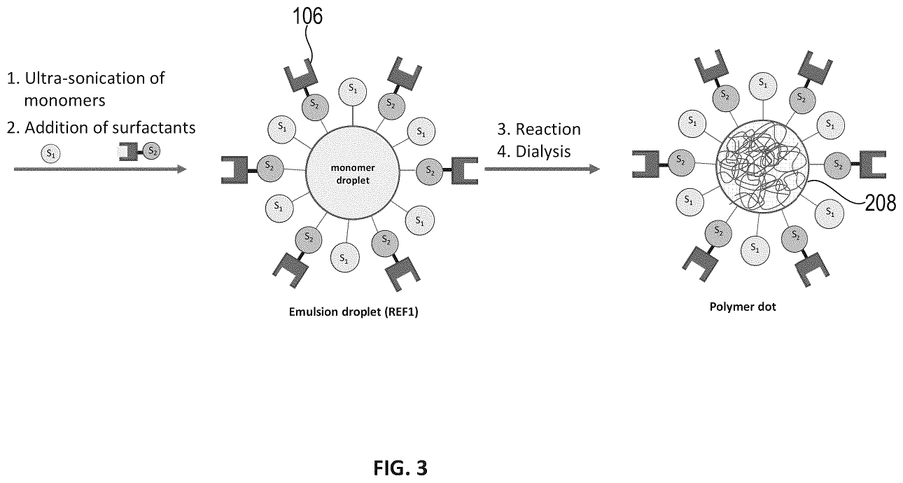

FIG. 3 is a cartoon drawing of an exemplary method of synthesizing a stabilized water-dispersible polymer dot with functionalized surfactants. In such an embodiment, the polymer dot can be synthesized via in situ emulsion polymerization. Monomers can be ultra-sonicated to produce monomer droplets. In such an example, ultra-sonication can be used during the reaction to target a particular droplet size range, for instance <5 nm. In some examples, the monomers may include dialdehyde monomer and/or di-cyanide monomer. Surfactants can be added (e.g., surfactants S₁ and S₂). In some examples, surfactant S₁ may be a polysorbate, for example polysorbate 80 (Tween® 80). Functional groups 106 can be added to the polymer dot via functionalized surfactants, for example via surfactants S₂. In such examples, the functional group 106 can be bound to the surfactant covalently. In some embodiments, the functionalized surfactant S₂ may include a polystyrene-based block co-polymer as described herein. In such embodiments, the polystyrene-based block copolymer may be telechelic and/or amphiphilic. A reaction then takes place to polymerize the monomers of the droplet. In such embodiments, the polymer dot 208 can be synthesized using a Knoevenagel condensation reaction. The resulting polymer dot 208 can optionally be size-filtered by dialysis.

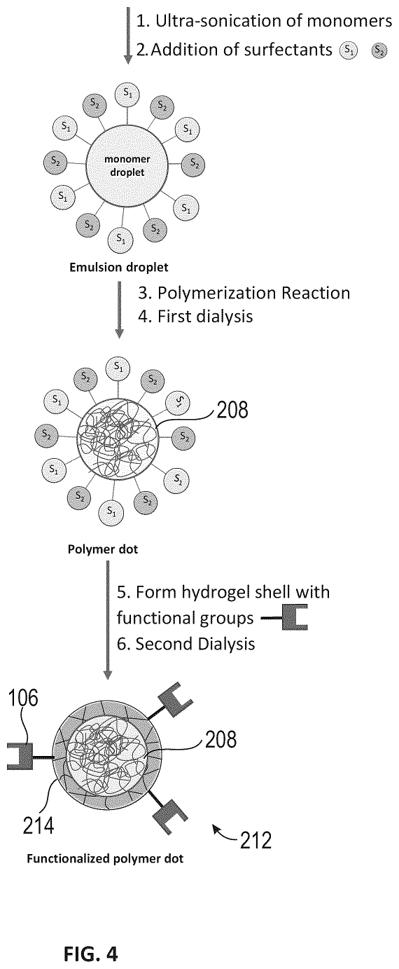

FIG. 4 is a cartoon drawing of an exemplary method of synthesizing a core-shell water-dispersible polymer dot. In such an embodiment, the polymer dot can be synthesized via in situ emulsion polymerization. Monomers can be ultra-sonicated to produce monomer droplets. In such an example, ultra-sonication can be used during the reaction to target a particular droplet size range, for instance <5 nm. In some examples, the monomers may include dialdehyde monomer and/or di-cyanide monomer. Surfactants can be added (e.g., surfactants S₁ and S₂). In some examples, surfactant S₁ may be a polysorbate, for example polysorbate 80. In some examples, surfactant S₂ may be sodium dodecyl sulfate (SDS). A reaction then takes place to polymerize the monomers of the droplet. In such embodiments, the polymer dot 208 can be synthesized using a Knoevenagel condensation reaction. The resulting polymer dot 208 can be optionally size-filtered by dialysis. A hydrogel shell 214 is formed over the polymer dot 208. In some examples, the hydrogel shell 214 may be a copolymer of one or more of N-isopropylacrylamide (NiPAM), bisacrylamide (bisAM), and acrylic acid. The hydrogel shell 214 can be functionalized by using one or more monomers containing reactive functional groups. In examples where the hydrogel shell 214 includes one or more of N-isopropylacrylamide, bisacrylamide, and acrylic acid, the functional moiety 106 may include a —COOH groups on the hydrogel surface that is capable of covalently bind the core-shell water-dispersible polymer dot with a nucleotide/oligonucleotide or polynucleotide with an amino functional group.

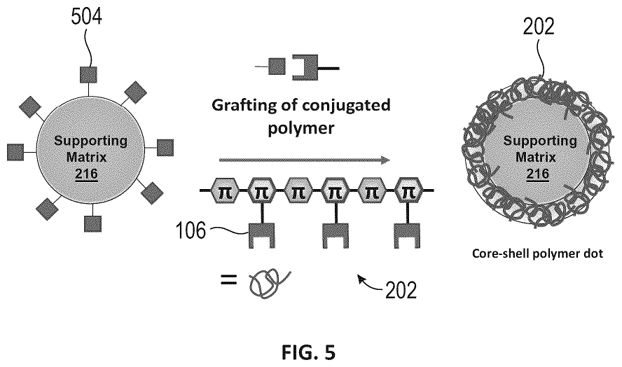

FIG. 5 is a cartoon drawing of an exemplary method of synthesizing a core-shell polymer dot. A supporting matrix 216 can include a plurality of reactive moieties 504. A conjugated polymer 202 can include a plurality of functional moieties 106 capable of binding the reactive moieties 504. The conjugated polymer can be grafted onto the surface of the supporting matrix. The resulting core-shell polymer dot includes a layer of functionalized water-soluble conjugated polymer 202 grafted on the surface of the supporting matrix 216. The polymer dot may optionally be size filtered by dialysis.

### Labeled Nucleotides

One aspect of the present disclosure relates to a labeled nucleotide, oligonucleotide or polynucleotide. The labeled nucleotide, oligonucleotide or polynucleotide comprises: a water-soluble conjugated polymer or a water-dispersible polymer dot covalently bound to the nucleotide, oligonucleotide or polynucleotide as a detectable label. The water-dispersible polymer dot may be in several different forms. For example, the water-dispersible polymer dot may include a conjugated polymer nanoparticle core, which is optionally stabilized with one or more surfactants. Functionalities may be introduced through either non-covalent interactions by using a functionalized surfactant, or coprecipitation with an amphiphilic polymer with functional groups (e.g., PS-PEG-COOH, PS-PEG-N₃, or styrene-maleic anhydride copolymers (PSMA)), or introduced by direct functionalization of the conjugated polymer side chain or backbone. In some other embodiments, the water-dispersible polymer dot is in the form of a core-shell polymer dot, either containing a polymer dot core and a functionalized hydrogel shell, or containing a supporting particle matrix and a functionalized conjugated polymer grafted (either by covalent bonding or non-covalent interactions) on the outer surface of the supporting particle matrix. In some embodiments, the core-shell water-dispersible polymer dot further comprises one or more surfactants on the surface of the conjugated polymer nanoparticle core. In further embodiments, the one or more surfactants comprises a water-soluble nonionic emulsifier (e.g., Polysorbate 80 (Tween® 80)) or an anionic surfactant (e.g., sodium dodecyl sulfate (SDS)), or a combination thereof.

In some embodiments, the water-soluble conjugated polymer or the water-dispersible polymer dot is covalently bounded to an unlabeled nucleotide, or an unlabeled nucleotide moiety at the terminal (e.g., 3′ terminal) of the oligonucleotide or polynucleotide. In some further embodiments, the water-soluble conjugated polymer or the water-dispersible polymer dot is covalently bounded to the unlabeled nucleotide or the unlabeled nucleotide moiety at the terminal (e.g., 3′ terminal) of the oligonucleotide or polynucleotide via a cleavable linker. In some embodiments, the oligonucleotide or polynucleotide is at least partially hybridized to a template polynucleotide immobilized on a solid support. In some embodiments, the solid support comprises an array of immobilized different template polynucleotides.

### 3′ Blocking Groups

The nucleotide described herein may also have a 3′ blocking group covalently attached to the deoxyribose sugar of the nucleotide. Various 3′ blocking group are disclosed in WO2002/029003, WO2004/018497 and WO2014/139596. For example, the blocking group may be azidomethyl (—CH₂N₃) or substituted azidomethyl (e.g., —CH(CHF₂)N₃ or CH(CH₂F)N₃), or allyl, each connecting to the 3′-oxygen atom of the deoxyribose moiety. In some embodiments, the 3′ blocking group is azidomethyl, forming 3′-OCH₂N₃ with the 3′ carbon of the ribose or deoxyribose.

Additional 3′ blocking groups are disclosed in U.S. Publication No. 2020/0216891 A1, which is incorporated by reference in its entirety. Non-limiting examples of the acetal blocking group

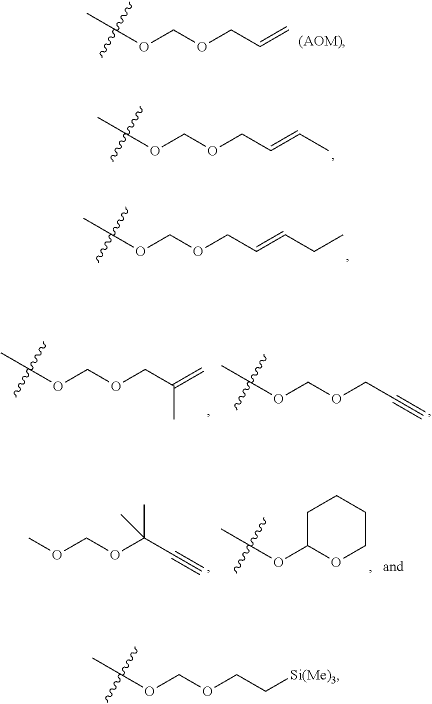

each covalently attached to the 3′ carbon of the deoxyribose.

### Deprotection of the 3′ Blocking Groups

In some embodiments, the 3′ hydroxy protecting group such as azidomethyl may be removed or deprotected by using a water-soluble phosphine reagent to generate a free 3′-OH. Non-limiting examples include tris(hydroxymethyl)phosphine (THMP), tris(hydroxyethyl) phosphine (THEP) or tris(hydroxypropyl)phosphine (THP or THPP). 3′-acetal blocking groups described herein may be removed or cleaved under various chemical conditions. For 3′ blocking groups that contain an allyl moiety, non-limiting cleaving condition includes a Pd(II) complex, such as Pd(OAc)₂ or allylPd(II) chloride dimer, in the presence of a phosphine ligand, for example tris(hydroxymethyl)phosphine (THMP), or tris(hydroxypropyl)phosphine (THP or THPP). For those blocking groups containing an alkynyl group (e.g., an ethynyl), they may also be removed by a Pd(II) complex (e.g., Pd(OAc)₂ or allyl Pd(II) chloride dimer) in the presence of a phosphine ligand (e.g., THP or THMP).

### Cleavable Linkers

As described herein, the reactive moiety or functional moiety of a nucleotide or a terminal unlabeled nucleotide moiety of the oligonucleotide or polynucleotide can be used to attach a conjugated polymer or polymer dot. In some embodiments, the reactant moiety of nucleotides described herein is covalently attached to the nucleobase of the nucleotide via a cleavable linker. Use of the term “cleavable linker” is not meant to imply that the whole linker is required to be removed. The cleavage site can be located at a position on the linker that ensures that part of the linker remains attached to the conjugated polymer and/or substrate moiety after cleavage. Cleavable linkers may be, by way of non-limiting example, electrophilically cleavable linkers, nucleophilically cleavable linkers, photocleavable linkers, cleavable under reductive conditions (for example disulfide or azide containing linkers), oxidative conditions, cleavable via use of safety-catch linkers and cleavable by elimination mechanisms. The use of a cleavable linker to attach the dye compound to a substrate moiety ensures that the label can, if required, be removed after detection, avoiding any interfering signal in downstream steps.

Useful linker groups may be found in PCT Publication No. WO2004/018493 (herein incorporated by reference), examples of which include linkers that may be cleaved using water-soluble phosphines or water-soluble transition metal catalysts formed from a transition metal and at least partially water-soluble ligands. In aqueous solution the latter form at least partially water-soluble transition metal complexes. Such cleavable linkers can be used to connect bases of nucleotides to labels such as the dyes set forth herein.

Particular linkers include those disclosed in PCT Publication No. WO2004/018493 (herein incorporated by reference) such as those that include moieties of the formulae:

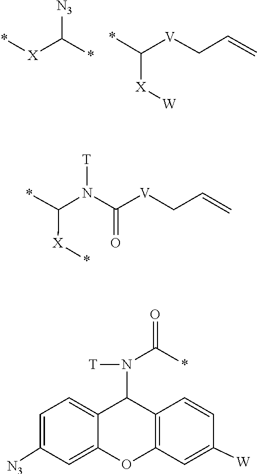

(wherein X is selected from the group comprising O, S, NH and NQ wherein Q is a C₁₋₁₀ substituted or unsubstituted alkyl group, Y is selected from the group comprising O, S, NH and N (allyl), T is hydrogen or a C₁-C₁₀ substituted or unsubstituted alkyl group and * indicates where the moiety is connected to the remainder of the nucleotide or nucleoside). In some aspect, the linkers connect the bases of nucleotides to labels.

Additional examples of linkers include those disclosed in U.S. Publication No. 2016/0040225 (herein incorporated by reference), such as those include moieties of the formulae:

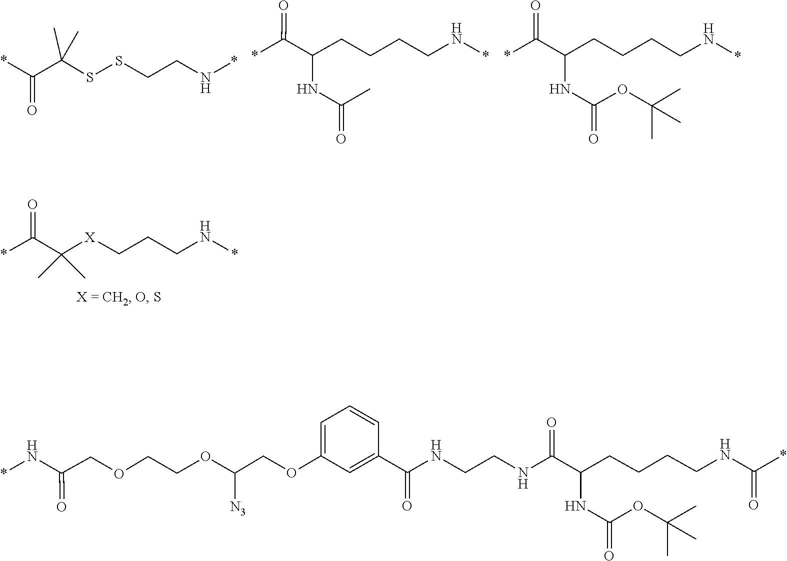

(wherein * indicates where the moiety is connected to the remainder of the nucleotide or nucleoside). The linker moieties illustrated herein may comprise the whole or partial linker structure between the nucleotides/nucleosides and the labels.

Additional examples of linkers are disclosed in U.S. Publication No. 2020/0216891 A1, which is incorporated by reference in its entirety:

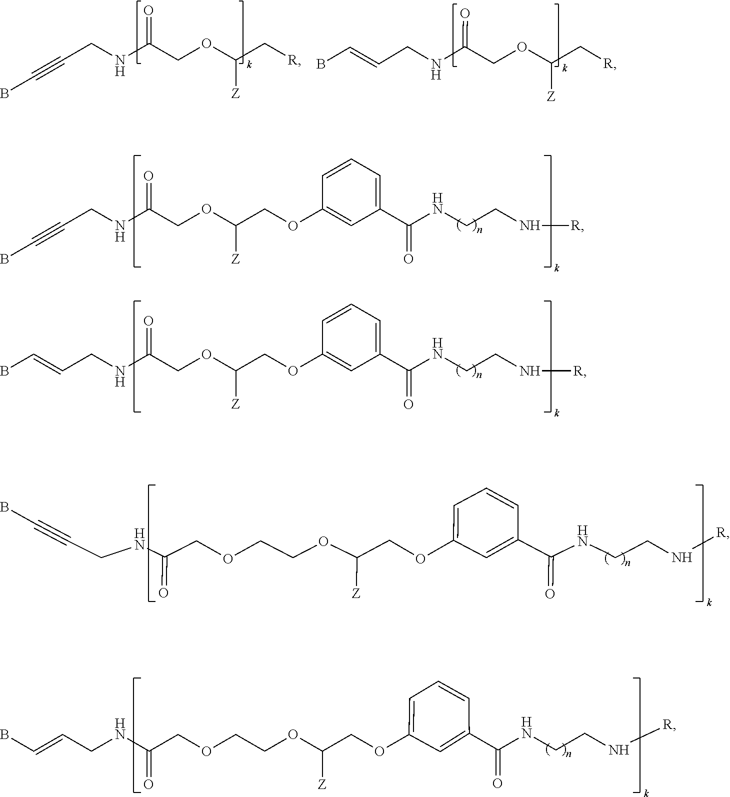

wherein B is a nucleobase; n is 1, 2, 3, 4, 5; k is 1; Z is —N₃ (azido), —O—C₁-C₆ alkyl, —O—C₂-C₆ alkenyl, or —O—C₂-C₆ alkynyl; and R is a reactive moiety or functional moiety described herein (that is capable of bind to the conjugated polymer or water-dispersible polymer dot via covalent bonding or non-covalent interactions), which may contain additional linker and/or spacer structure. One of ordinary skill in the art understands that the first, the second, or the third functional moiety described herein is covalently bound to the linker by reacting a functional group of the functional moiety containing compound (e.g., carboxyl) with a functional group of the linker (e.g., amino) to form an amide bond. In one embodiment, the cleavable linker comprises

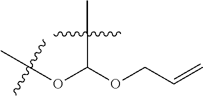 [SMILES: *OC(*)OCC=C]

(“AOL” linker moiety) where Z is —O-allyl. For the purpose of the present disclosure, the nucleotide may contain multiple cleavable linkers repeating units (e.g., k is 1, 2, 3, 4 5, 6, 7, 8, 9 or 10).

The reactive moiety may be attached to any position on the nucleotide base, for example, through a linker. In particular embodiments, Watson-Crick base pairing can still be carried out for the resulting analog. Particular nucleobase labeling sites include the C5 position of a pyrimidine base or the C7 position of a 7-deaza purine base. As described above a linker group may be used to covalently attach a dye to the nucleotide.

In particular embodiments, the unlabeled nucleotide may be enzymatically incorporable and enzymatically extendable. Accordingly, a linker moiety may be of sufficient length to connect the nucleotide to the compound such that the compound does not significantly interfere with the overall binding and recognition of the nucleotide by a nucleic acid replication enzyme. Thus, the linker can also comprise a spacer unit, such as one or more PEG unit(s)

(—OCH₂CH₂—)ₙ, where n is an integer of 1-20, for example 1, 2, 3, 4, 5, 6, 7, 8, 9, 10, 11, 12, 13, 14 or 15. The spacer distances, for example, the nucleotide base from a cleavage site or label.

In yet another alternative embodiment, there is no blocking group on the 3′ carbon of the pentose sugar and the labeling reagent attached to the base via a linker, for example, can be of a size or structure sufficient to act as a block to the incorporation of a further nucleotide. Thus, the block can be due to steric hindrance or can be due to a combination of size, charge and structure, whether or not the dye is attached to the 3′ position of the sugar.

The use of a blocking group allows polymerization to be controlled, such as by stopping extension when an unlabeled nucleotide is incorporated. If the blocking effect is reversible, for example, by way of non-limiting example by changing chemical conditions or by removal of a chemical block, extension can be stopped at certain points and then allowed to continue.

In particular embodiments, the length of the linker between a reactive moiety and a guanine base can be altered, for example, by introducing a polyethylene glycol spacer group, thereby increasing the fluorescence intensity compared to the same fluorophore attached to the guanine base through other linkages known in the art. Exemplary linkers and their properties are set forth in PCT Publication No. WO 2007/020457 (herein incorporated by reference). The design of linkers, and especially their increased length, can allow for improvements in the brightness of fluorophores (e.g., conjugated polymers or polymer dots) attached to the guanine bases of guanosine nucleotides when incorporated into polynucleotides such as DNA. Thus, when conjugated polymers or polymer dots are for use in any method of analysis which requires detection of a fluorescent dye label attached to a guanine-containing nucleotide, it may be advantageous if the linker comprises a spacer group of formula —((CH₂)₂O)ₙ—, wherein n is an integer between 2 and 50, as described in WO 2007/020457.

Nucleosides and nucleotides may include linkers on the sugar or nucleobase. As known in the art, a “nucleotide” consists of a nitrogenous base, a sugar, and one or more phosphate groups. In RNA, the sugar is ribose and in DNA is a deoxyribose, i.e., a sugar lacking a hydroxy group that is present in ribose. The nitrogenous base is a derivative of purine or pyrimidine. The purines are adenine (A) and guanine (G), and the pyrimidines are cytosine (C) and thymine (T) or in the context of RNA, uracil (U). The C-1 atom of deoxyribose is bonded to N-1 of a pyrimidine or N-9 of a purine. A nucleotide is also a phosphate ester of a nucleoside, with esterification occurring on the hydroxy group attached to the C-3 or C-5 of the sugar. Nucleotides are usually mono, di- or triphosphates.

Although the base is usually referred to as a purine or pyrimidine, the skilled person will appreciate that derivatives and analogues are available which do not alter the capability of the nucleotide or nucleoside to undergo Watson-Crick base pairing. “Derivative” or “analogue” means a compound or molecule whose core structure is the same as, or closely resembles that of a parent compound but which has a chemical or physical modification, such as, for example, a different or additional side group, which allows the derivative nucleotide or nucleoside to be linked to another molecule. For example, the base may be a deazapurine. In particular embodiments, the derivatives should be capable of undergoing Watson-Crick pairing. “Derivative” and “analogue” also include, for example, a synthetic nucleotide or nucleoside derivative having modified base moieties and/or modified sugar moieties. Such derivatives and analogues are discussed in, for example, Scheit, Nucleotide analogs (John Wiley & Son, 1980) and Uhlman et al., Chemical Reviews 90:543-584, 1990. Nucleotide analogues can also comprise modified phosphodiester linkages including phosphorothioate, phosphorodithioate, alkyl-phosphonate, phosphoranilidate, phosphoramidate linkages and the like.

Non-limiting exemplary unlabeled nucleotides as described herein include:

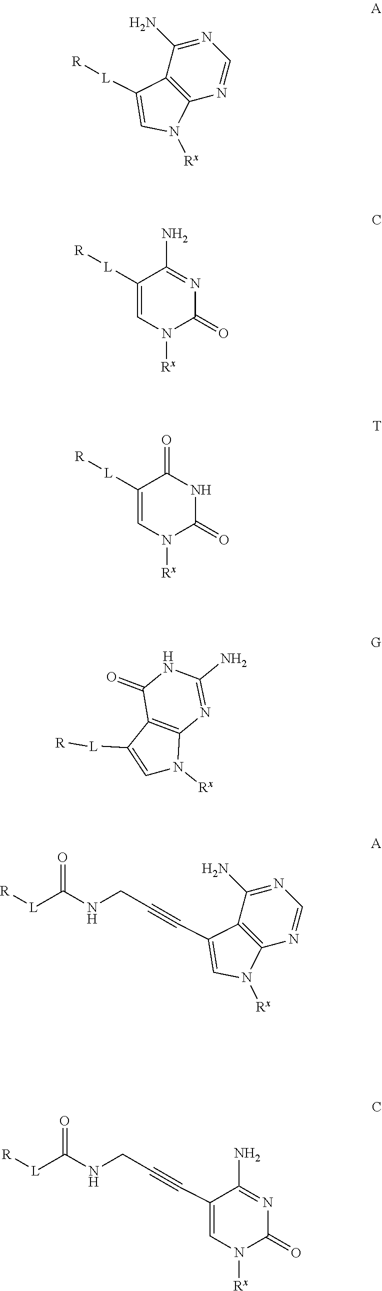

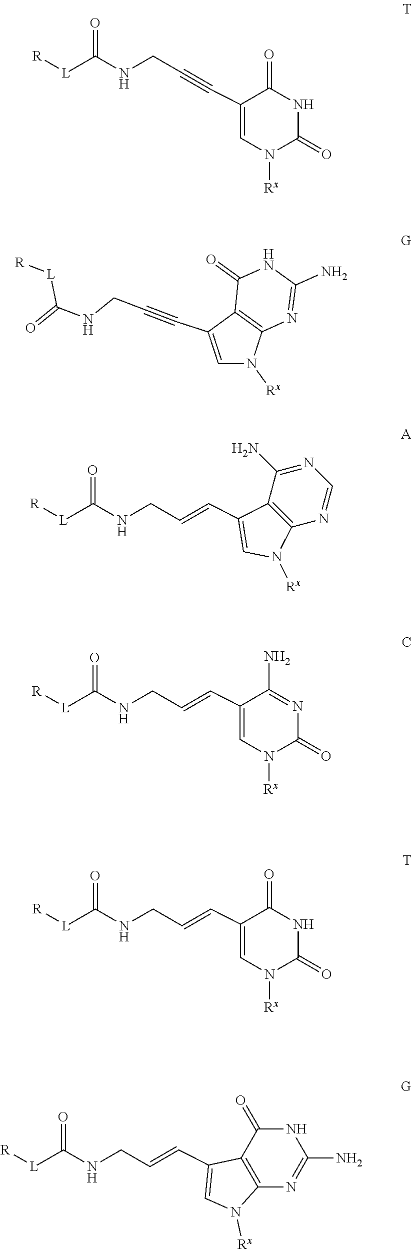

wherein L represents a linker, including a cleavable linker described herein; R^{x} represents a ribose or deoxyribose moiety as described above, or a ribose or deoxyribose moiety with the 5′ position substituted with mono-, di- or tri-phosphates; R represents the reactive moiety described herein.

In some embodiments, non-limiting exemplary unlabeled nucleotide containing a hapten moiety covalently attached via a cleavable linker are shown below:

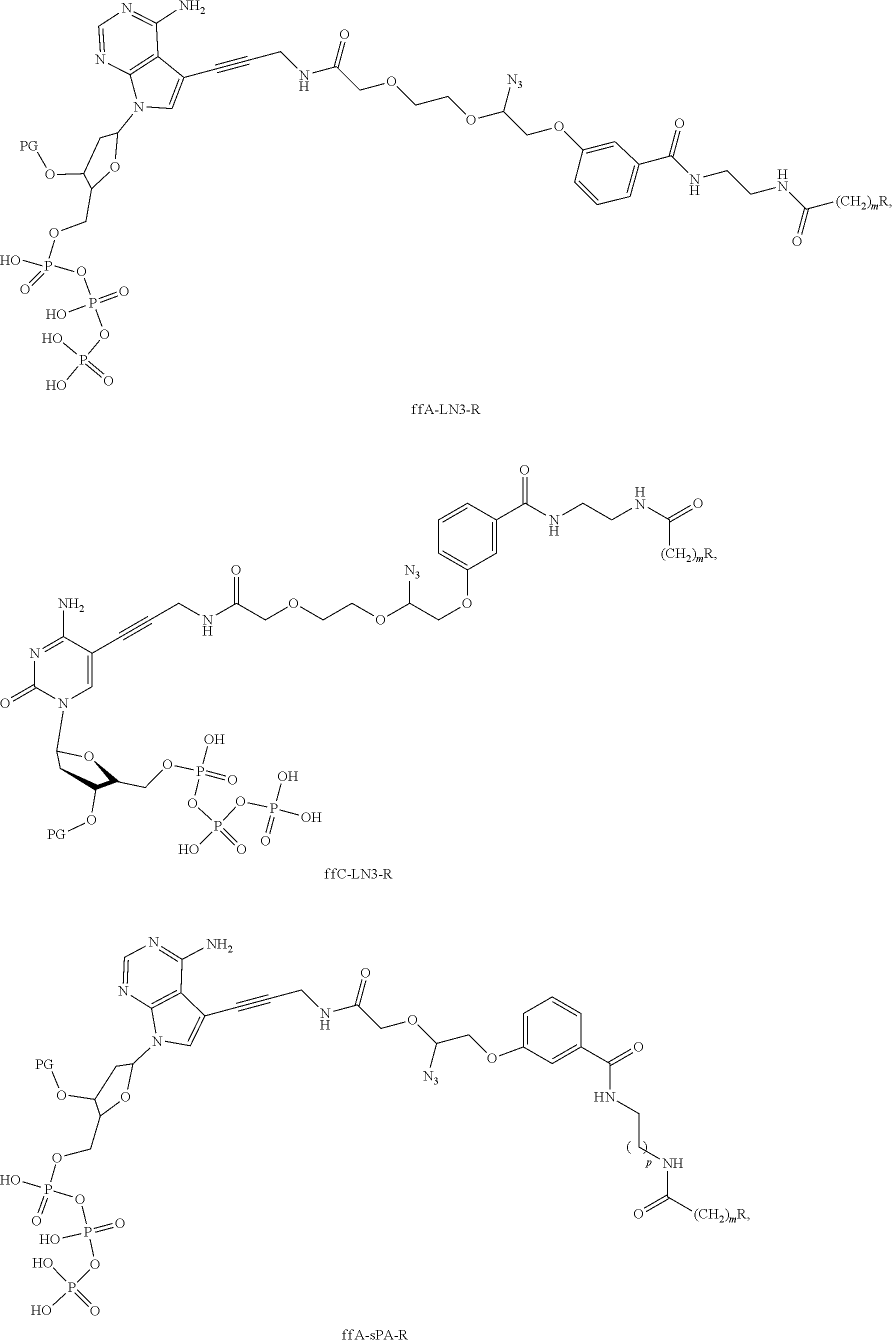

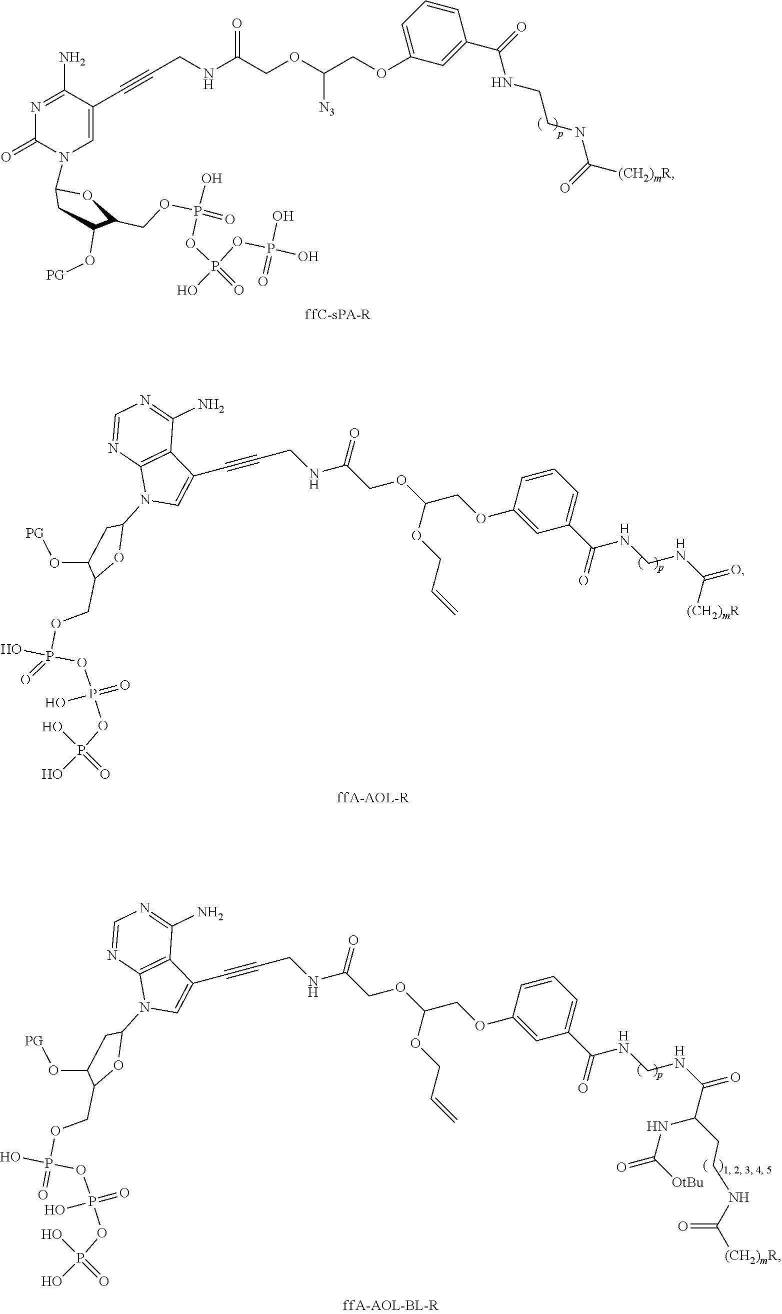

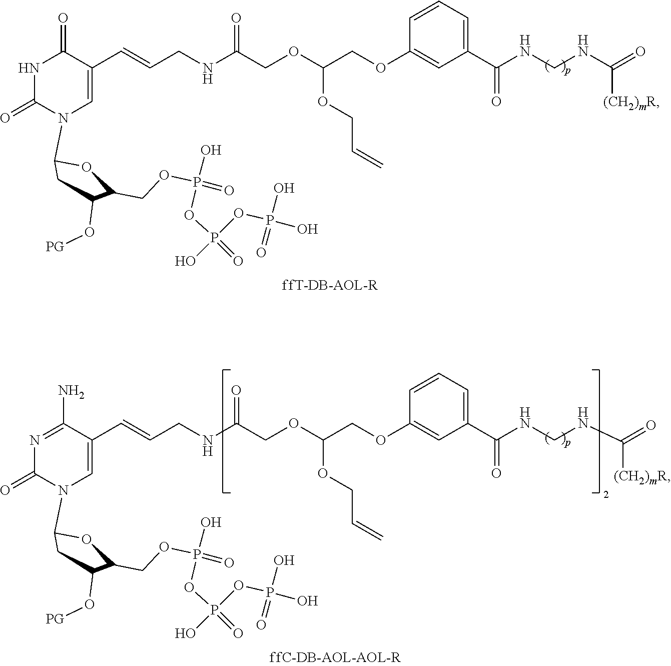

wherein PG stands for the 3′ blocking groups described herein; p is an integer of 1, 2, 3, 4, 5, 6, 7, 8, 9, or 10; and m is 0, 1, 2, 3, 4, or 5. In one embodiment, —O-PG is AOM. In another embodiment, —O-PG is —O-azidomethyl. In one embodiment, m is 5. In another embodiment, m is 0. In another embodiment, m is 2. In some further embodiments, p is 1, 2, 3, 4 or 5.

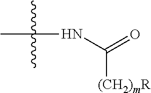

refers to the connection point of the first/second/third functional moiety with the cleavable linker as a result of a reaction between an amino group of the linker moiety and the carboxyl group of the first/second/third functional moiety. In further embodiments, the nucleotide may be attached to the hapten moiety via more than one of the same cleavable linkers (such as LN3-LN3, sPA-SPA, AOL-AOL). In other embodiments, the nucleotide may be attached to the hapten moiety via two or more different cleavable linkers (such sPA-LN3, SPA-SPA-LN3, SPA-LN3-LN3, etc.). In addition, the linker may further include additional PEG spacers as described herein, for example, between R and
—(CH₂)ₘ—. In any embodiments of the labeled nucleotide described herein, the nucleotide is a nucleotide triphosphate. In further embodiments, the nucleotide has a 2′ deoxyribose.

### Kits

One aspect of the present disclosure relates to a kit of sequencing application, comprising: an incorporation mixture composition comprising one or more of four different types of nucleotides (A, C, G and T or U) each comprising a 3′ blocking group, wherein a first type of nucleotide is a nucleotide labelled with a water-soluble conjugated polymer or a water-dispersible polymer dot as described herein.

Another aspect of the present disclosure relates to a kit for sequencing application, comprising:
an incorporation mixture composition comprising one or more of four different types of nucleotides (A, C, G and T or U) each comprising a 3′ blocking group, wherein a first type of unlabeled nucleotide comprises a first reactive moiety covalently attached thereto; and
a first labeling reagent comprising a water-soluble conjugated polymer or a water-dispersible polymer dot, each having a first functional moiety that is capable of reacting specifically with the first reactive moiety of the first type of unlabeled nucleotide to form bonding (either via covalent bonding or non-covalent interaction). In some embodiments, the first reactive moiety of the first type of unlabeled nucleotide is capable of forming covalent bonding with the first functional moiety of the first labeling reagent via a biorthogonal reaction selected from the group consisting of a [3+2] dipolar cycloaddition, a Diels-Alder cycloaddition, a [4+1] cycloaddition, a phosphine ligation, or condensation with 2-acylphenyl boronic acid.

In some embodiments of the kit described herein, the first reactive moiety is covalently attached to the nucleobase of the first type of unlabeled nucleotide via a cleavable linker. In some embodiments, the first labeling reagent comprises the water-soluble conjugated polymer having one or more repeating unit moieties selected from the group consisting of:

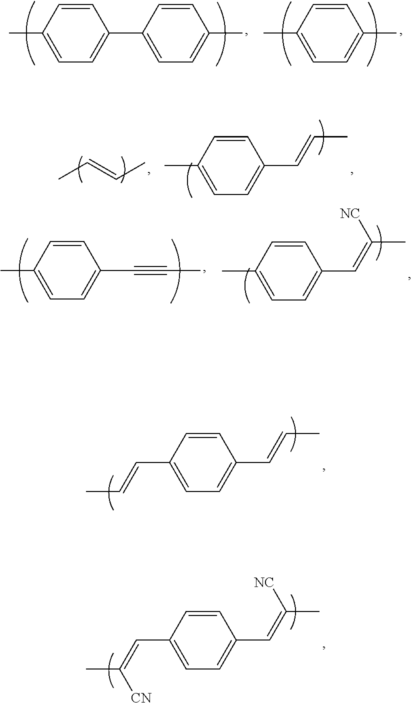

a five membered heteroarylene containing one, two or three heteroatoms selected from the group consisting of N, O and S,

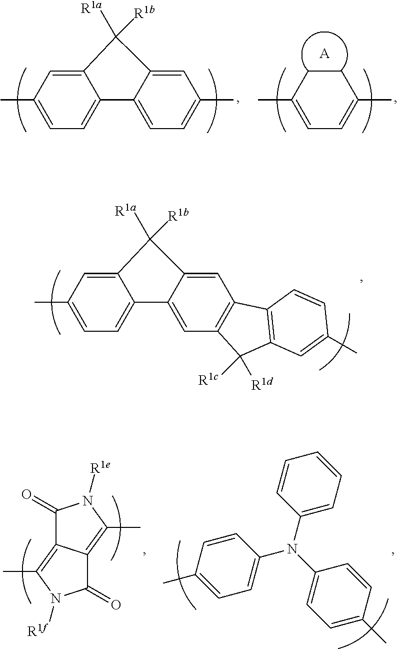

and combinations thereof;
wherein each of R^{1a}, R^{1b}, R^{1c}, R^{1d}, R^{1e} and R^{1f} is independently H, unsubstituted C₁-C₁₂ alkyl, C₁-C₁₂ alkyl substituted with one or more water solubilizing groups, or 2 to 20 membered heteroalkyl (e.g., 2-20 membered heteroalkyl comprising 2, 3, 4, 5, or 6 polyethylene glycol unit (—OCH₂CH₂));
ring A is a five or six membered heteroaryl containing one, two or three heteroatoms selected from the group consisting of N, O and S, and
wherein each structure is optionally substituted with one or more electron donating groups, one or more electron withdrawing groups, one or more functional moiety R^{F}, or one or more water solubilizing group R^{S}, or combinations thereof. In some embodiments, the electron donating groups comprise a C₁-C₁₂ alkoxy, for example, —OC₈H₁₇. In some embodiments, electron withdrawing group includes —CN. In some embodiments, the water solubilizing groups are selected from the group consisting of phosphate;

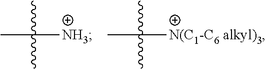

wherein each C₁-C₆ alkyl is optionally substituted with one or more substituents selected from phosphate,

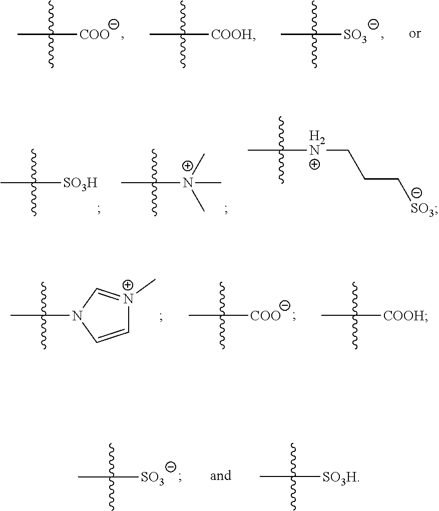

In some examples, the functional moiety of the water-soluble conjugated polymer is selected from the group consisting of azido, amino, a reactive ester,

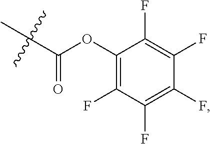 [SMILES: *C(=O)Oc1c(F)c(F)c(F)c(F)c1F]

unsubstituted or substituted 1,2,4,5-tetrazinyl,

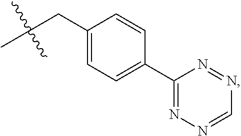 [SMILES: *Cc1ccc(-c2nncnn2)cc1]

unsubstituted or substituted dibenzocyclooctyne (DBCO)

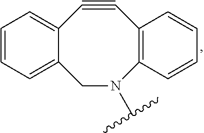 [SMILES: *N1Cc2ccccc2C#Cc2ccccc21]

unsubstituted or substituted bicyclo[6.1.0]nonynyl (BCN),

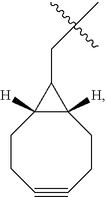

transcyclooctenyl (TCO), and

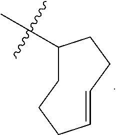 [SMILES: *C1CC/C=C/CCC1]

In some other embodiments of the kit described herein, the first labeling reagent comprises a water-dispersible polymer dot, wherein the water-dispersible polymer dot comprises a conjugated polymer nanoparticle and one or more functionalized surfactants attached to the surface of the nanoparticle through non-covalent interaction. In some further embodiments, the conjugated polymer comprises one or more repeating unit moieties having a structure selected from the group consisting of

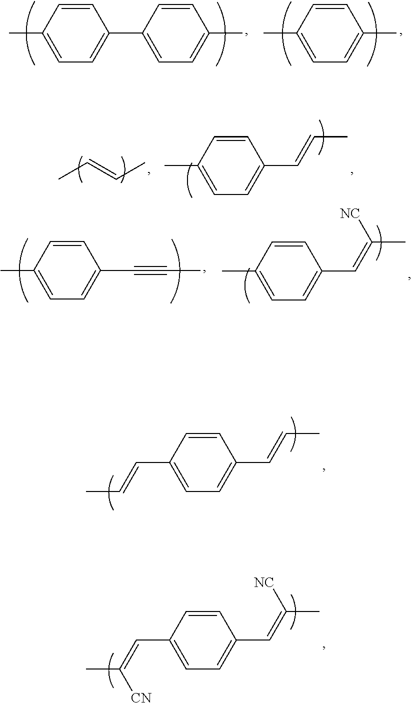

a five membered heteroarylene containing one, two or three heteroatoms selected from the group consisting of N, O and S,

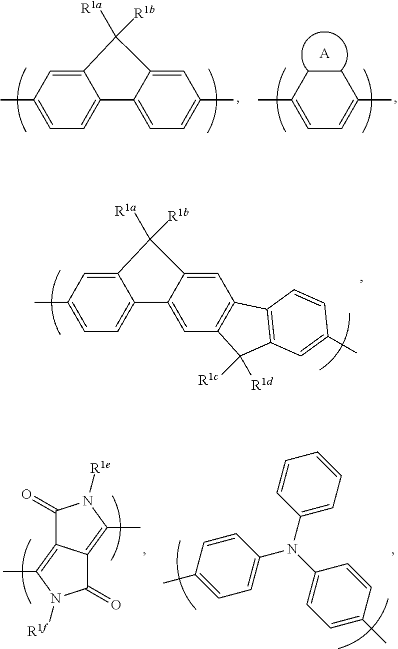

and combinations thereof;
wherein each of R^{1a}, R^{1b}, R^{1c}, R^{1d}, R^{1e} and R^{1f} is independently H, unsubstituted C₁-C₁₂ alkyl, C₁-C₁₂ alkyl substituted with one or more water solubilizing groups, or 2 to 20 membered heteroalkyl;
ring A is a five or six membered heteroaryl containing one, two or three heteroatoms selected from the group consisting of N, O and S, and
wherein each structure is optionally substituted with one or more electron donating groups, one or more electron withdrawing groups, one or more functional moiety R^{F}, or one or more water solubilizing group R^{S}, or combinations thereof. In some embodiments, the electron donating groups comprise a C₁-C₁₂ alkoxy, for example —OC₈H₁₇. In some embodiments, electron withdrawing group includes —CN. In some further embodiments, R^{S} is selected from the group consisting of phosphate;

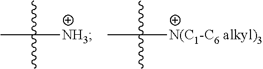

(wherein each C₁-C₆ alkyl is optionally substituted with one or more substituents selected from phosphate,

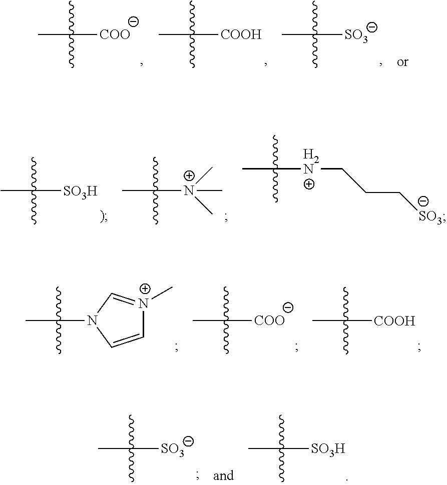

In some embodiments, the one or more functionalized surfactants comprises an amphiphilic block copolymer or a water-soluble nonionic emulsifier (e.g., Polysorbate 80 (Tween® 80)), or a combination thereof. In some further embodiments, the amphiphilic block copolymer comprises

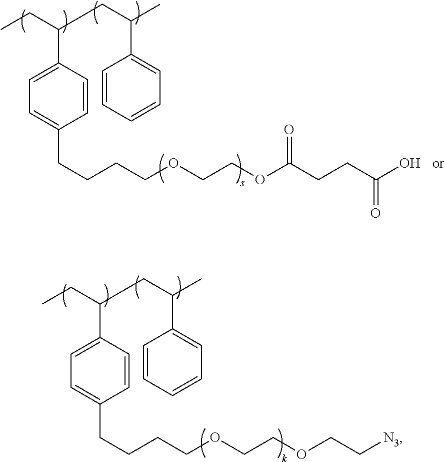

each of s and k is independently an integer from 1 to 10,000.

In some additional embodiments of the kit described herein, the first labeling reagent comprises a core-shell water-dispersible polymer dot, wherein the core-shell water-dispersible polymer dot comprises a conjugated polymer nanoparticle core, and a hydrogel shell. In some further embodiments, the core-shell water-dispersible polymer dot further comprises one or more surfactants on the surface of the conjugated polymer nanoparticle core. In yet further embodiments, the one or more surfactants comprises a water-soluble nonionic emulsifier (e.g., Polysorbate 80 (Tween® 80)) or an anionic surfactant (e.g., sodium dodecyl sulfate (SDS)), or a combination thereof. In some further embodiments, wherein the hydrogel shell comprises an acrylamide hydrogel. In yet a further embodiment, the acrylamide hydrogel is a copolymer of N-isopropylacrylamide (NiPAM), bisacrylamide (bisAM), and acrylic acid (AAc). In other embodiments, the first labeling reagent comprises a core-shell water-dispersible polymer dot, wherein the core-shell water-dispersible polymer dot comprises a water-soluble conjugated polymer coated on a supporting particle matrix core as described herein. In some further examples, the water-soluble conjugated polymer is bonded to the supporting particle matrix core via covalent bonding or non-covalent bonding.

In some embodiments, the functional moiety of the water-dispersible polymer dot is selected from the group consisting of azido, amino, a reactive ester,

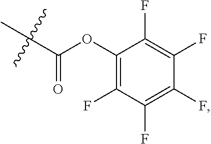 [SMILES: *C(=O)Oc1c(F)c(F)c(F)c(F)c1F]

unsubstituted or substituted 1,2,4,5-tetrazinyl,

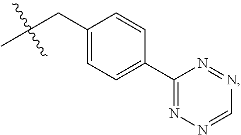 [SMILES: *Cc1ccc(-c2nncnn2)cc1]

unsubstituted or substituted DBCO,

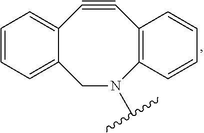 [SMILES: *N1Cc2ccccc2C#Cc2ccccc21]

unsubstituted or substituted BCN,

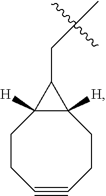

unsubstituted or substituted TCO, and

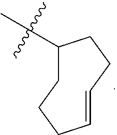 [SMILES: *C1CC/C=C/CCC1]

In some embodiments of the functional moiety described herein, an azido may include primary azide such as

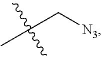 [SMILES: *CN=[N+]=[N-]]

aryl azide such as

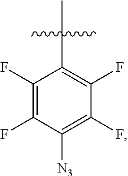 [SMILES: *c1c(F)c(F)c(N=[N+]=[N-])c(F)c1F]

or tertiary azide such as

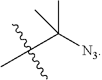 [SMILES: *C(C)(C)N=[N+]=[N-]]

1,2,4,5-tetrazine may include phenyl tetrazine

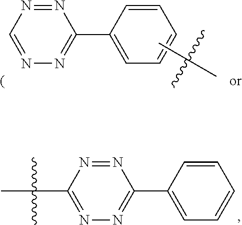

wherein the phenyl ring may be optionally substituted), pyrimidyl tetrazine

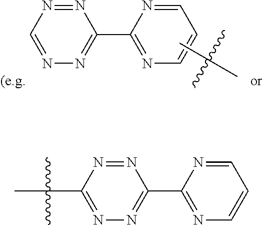

wherein the pyrimidyl ring is optionally substituted), methyl tetrazine

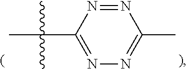 [SMILES: *c1nnc(C)nn1]

pyridyl tetrazine

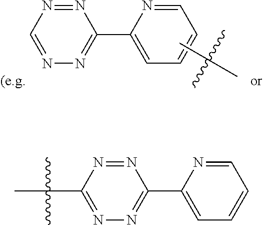

where the pyridyl ring is optionally substituted), t-butyl tetrazine

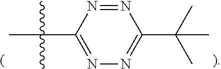 [SMILES: *c1nnc(C(C)(C)C)nn1]

In some other embodiments, the first reactive moiety of the first type of unlabeled nucleotide is attached to the first labeling reagent via non-covalent interactions. For example, a functional moiety

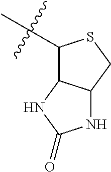 [SMILES: *C1SCC2NC(=O)NC21]

of the unlabeled nucleotide forms noncovalent bonding with an avidin (e.g., streptavidin) moiety of first labeling reagent, or vice versa.

In some such embodiments of the kit described herein, each of the second type of nucleotide and the third type of nucleotide is labeled. In some other embodiments, the second type of nucleotide is a second type of unlabeled nucleotide having a second reactive moiety covalently attached to the second type of unlabeled nucleotide, and the kit further comprises a second labeling reagent comprising a second detectable label and a second functional moiety that is capable of reacting specifically with the second reactive moiety to form covalent or noncovalent bonding, and wherein the fourth type of unlabeled nucleotide is not capable of reacting with either the first labeling reagent or the second labeling reagent. In some such embodiments, the third type of nucleotide is labeled. In some other such embodiments, the third type of nucleotide is a mixture of a third type of unlabeled nucleotide having a first reactive moiety covalently attached to the third type of unlabeled nucleotide and a third type of unlabeled nucleotide having a second reactive moiety covalently attached to the third type of unlabeled nucleotide. In some other such embodiments, the third type of nucleotide is a third type of unlabeled nucleotide having a third reactive moiety covalently attached to the third type of unlabeled nucleotide, and the kit further comprises a third labeling reagent comprising a third detectable label and a third functional moiety that is capable of reacting specifically to the third reactive moiety of the third type of unlabeled nucleotide to form covalent or noncovalent bonding, and wherein the fourth type of unlabeled nucleotide is not capable of reacting with any one of the first labeling reagent, the second labeling reagent, or the third labeling reagent. The second functional moiety and the second reactive moiety can undergo a biorthogonal reaction as described herein with respect to the reaction between the first functional moiety and the first reactive moiety. The third functional moiety and the third reactive moiety can undergo a biorthogonal reaction as described herein with respect to the reaction between the first functional moiety and the first reactive moiety. For example, the kit may comprise:
a first type of unlabeled nucleotide having a first reactive moiety covalently attached to the first type of unlabeled nucleotide;
a second type of unlabeled nucleotide having a second reactive moiety covalently attached to the second type of unlabeled nucleotide;
a mixture of a third type of unlabeled nucleotide having a first reactive moiety covalently attached to the third type of unlabeled nucleotide and a third type of unlabeled nucleotide having a second reactive moiety covalently attached to the third type of unlabeled nucleotide;
a fourth type of unlabeled nucleotide;
a first labeling reagent comprising a first functional moiety that is capable of reacting specifically with the first functional moiety to form covalent bonding; and
a second labeling reagent comprising a second functional moiety that is capable of reacting specifically with the second functional moiety to form covalent bonding;
wherein the fourth type of unlabeled nucleotide is not capable of reacting with the first labeling reagent and the second labeling reagent.

In some embodiments, the kit further comprises a DNA polymerase and one or more buffer compositions. In some embodiments, the incorporation mixture composition further comprises a DNA polymerase, such as a mutant of 9°N polymerase disclosed in WO 2005/024010, U.S. Publication Nos. 2020/0131484 A1, 2020/0181587 A1, and 2024/0141427 A1, each of which is incorporated by reference in its entirety.

In some embodiments, the four different types of nucleotides are distinguishable using a single light source (e.g., a blue light source having a wavelength from about 450 nm to about 460 nm). In some other embodiments, the four different types of nucleotides are distinguishable using two light sources operating at two different wavelengths. For example, one light source has a wavelength of about 450 nm to about 460 nm, and the other light source has a wavelength of about 520 nm to about 540 nm.

### Methods of Sequencing

One aspect of the present disclosure relates to a method of determining the sequences of a plurality of different target polynucleotides, comprising:
(a) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(b) contacting the solid support with an aqueous solution comprising DNA polymerase and one or more of four different types of unlabeled nucleotides under conditions suitable for DNA polymerase-mediated primer extension, and incorporating one type of nucleotides into the sequencing primers to produce extended copy polynucleotides; wherein
each of the four types of nucleotides comprises a 3′ blocking group; and
the incorporation mixture comprises a first type of unlabeled nucleotide having a first reactive moiety covalently attached to the first type of unlabeled nucleotide;
(c) contacting the extended copy polynucleotides with a first labeling reagent comprising a water-soluble conjugated polymer or a water-dispersible polymer dot comprising a functional moiety that reacts specifically with the first reactive moiety of the first type of unlabeled nucleotides to form covalent or non-covalent bonding;
(d) imaging the solid support and performing one or more fluorescent measurements of the extended copy polynucleotides; and
(e) removing the 3′ blocking group of the incorporated nucleotides.

In some embodiments, the first reactive moiety is covalently attached to the first type of unlabeled nucleotide via a cleavable linker. In some embodiments, the first labeling reagent comprises the water-soluble conjugated polymer, the water-soluble conjugated polymer comprises one or more repeating unit moieties selected from the group consisting of:

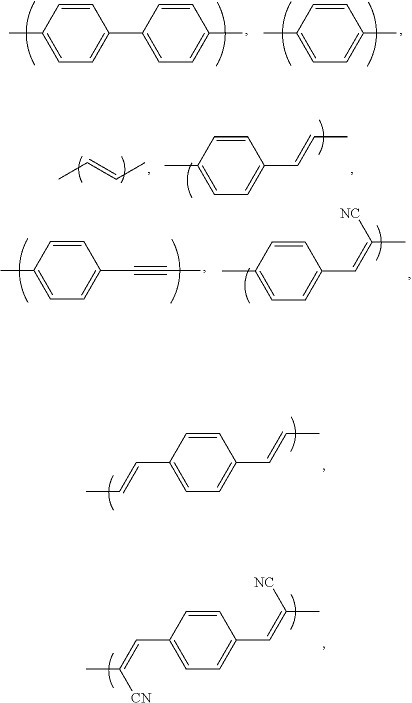

a five membered heteroarylene containing one, two or three heteroatoms selected from the group consisting of N, O and S,

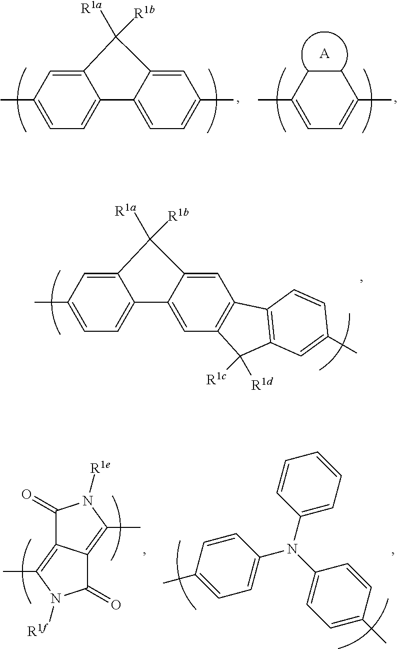

and combinations thereof;
wherein each of R^{1a}, R^{1b}, R^{1c}, R^{1d}, R^{1e} and R^{1f} is independently H, unsubstituted C₁-C₁₂ alkyl, C₁-C₁₂ alkyl substituted with one or more water solubilizing groups, or 2 to 20 membered heteroalkyl (e.g., 2-20 membered heteroalkyl comprising 2, 3, 4, 5, or 6 polyethylene glycol unit (—OCH₂CH₂));
ring A is a five or six membered heteroaryl containing one, two or three heteroatoms selected from the group consisting of N, O and S, and
wherein each structure is optionally substituted with one or more electron donating groups, one or more electron withdrawing groups, one or more functional moiety R^{F}, or one or more water solubilizing group R^{S}, or combinations thereof. In some further embodiments, the water solubilizing groups are selected from the group consisting of phosphate;

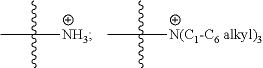

(wherein each C₁-C₆ alkyl is optionally substituted with one or more substituents selected from phosphate,

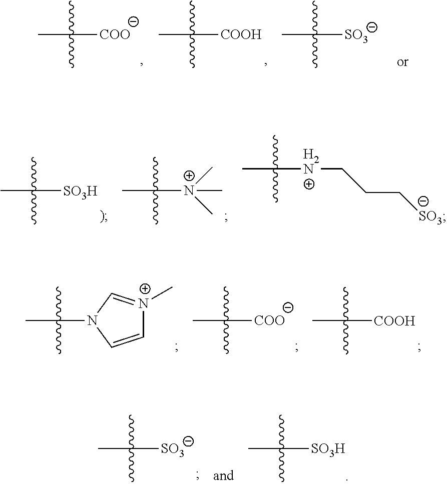

In some embodiments, the functional moiety of the water-soluble conjugated polymer is azido, amino, a reactive ester,

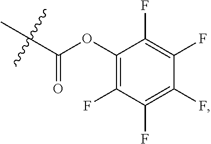 [SMILES: *C(=O)Oc1c(F)c(F)c(F)c(F)c1F]

unsubstituted or substituted 1,2,4,5-tetrazinyl,

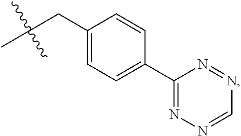 [SMILES: *Cc1ccc(-c2nncnn2)cc1]

unsubstituted or substituted DBCO,

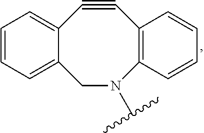 [SMILES: *N1Cc2ccccc2C#Cc2ccccc21]

unsubstituted or substituted BCN,

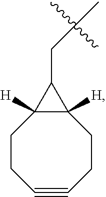

TCO, or

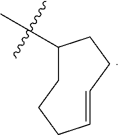 [SMILES: *C1CC/C=C/CCC1]

More specific examples of the water-soluble conjugated polymer include:

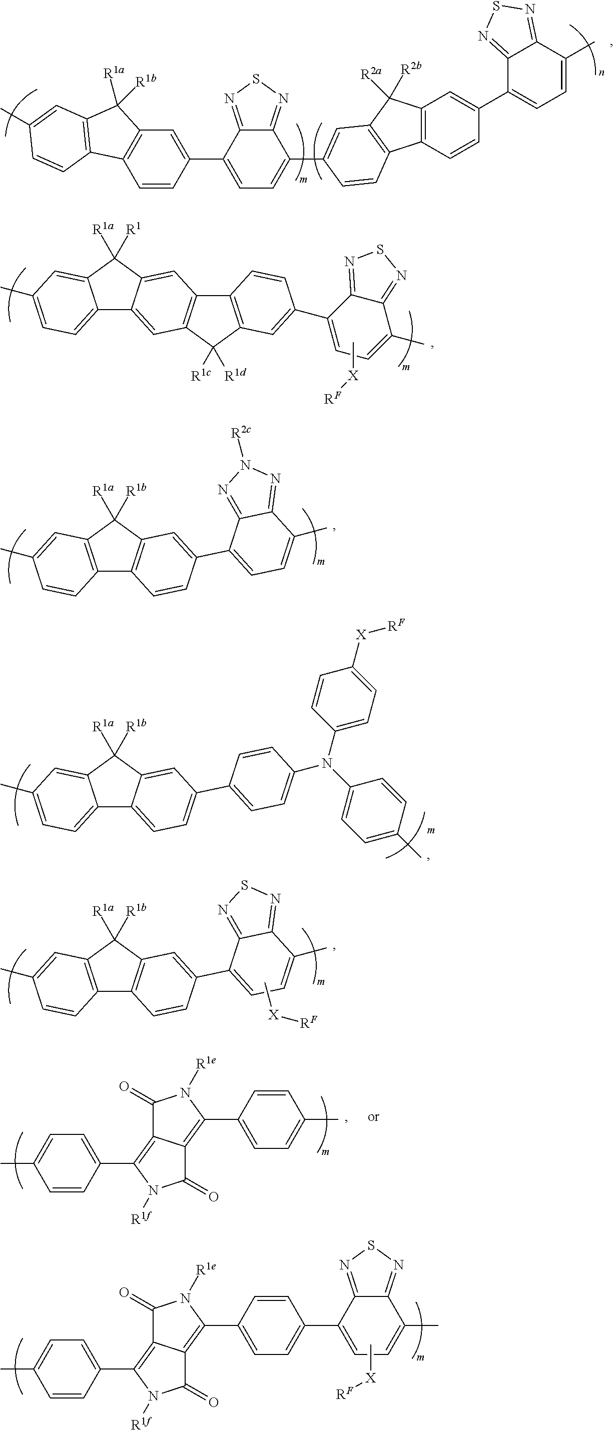

as described herein.

In some embodiments, the first labeling reagent comprises the water-dispersible polymer dot, wherein the water-dispersible polymer dot comprises a conjugated polymer nanoparticle and one or more functionalized surfactants attached to the surface of the nanoparticle through non-covalent interaction. In some further embodiments, the conjugated polymer comprises one or more repeating unit moieties having a structure selected from the group consisting of

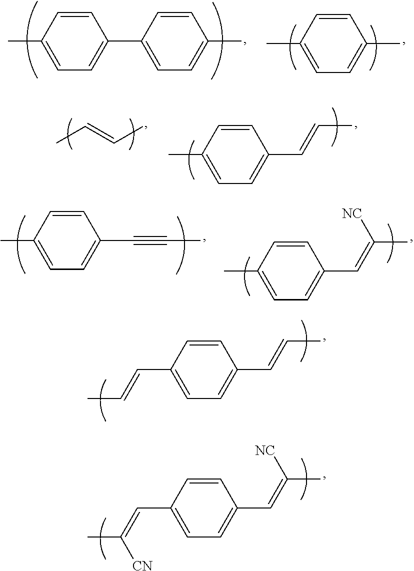

a five membered heteroarylene containing one, two or three heteroatoms selected from the group consisting of N, O and S,

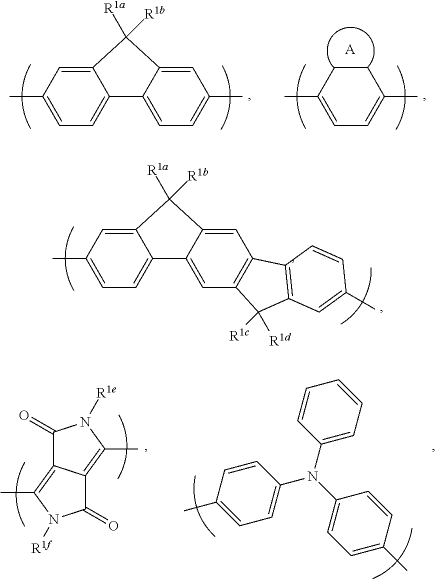

and combinations thereof;
wherein each of R^{1a}, R^{1b}, R^{1c}, R^{1d}, R^{1e} and R^{1f} is independently H, unsubstituted C₁-C₁₂ alkyl, C₁-C₁₂ alkyl substituted with one or more water solubilizing groups, or 2 to 20 membered heteroalkyl;
ring A is a five or six membered heteroaryl containing one, two or three heteroatoms selected from the group consisting of N, O and S, and
wherein each structure is optionally substituted with one or more electron donating groups, one or more electron withdrawing groups, one or more functional moiety R^{F}, or one or more water solubilizing group R^{S}, or combinations thereof. In some embodiments, the electron donating groups comprise a C₁-C₁₂ alkoxy, for example —OC₈H₁₇. In some embodiments, electron withdrawing group includes —CN. In some further embodiments, R^{S} is selected from the group consisting of phosphate;

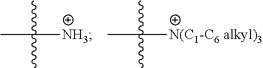

(wherein each C₁-C₆ alkyl is optionally substituted with one or more substituents selected from phosphate,

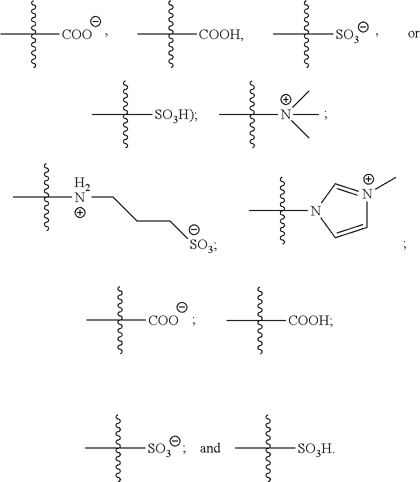

In some embodiments, the functional moiety of the water-dispersible polymer dot is azido, amino, a reactive ester,

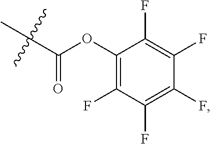 [SMILES: *C(=O)Oc1c(F)c(F)c(F)c(F)c1F]

unsubstituted or substituted 1,2,4,5-tetrazinyl,

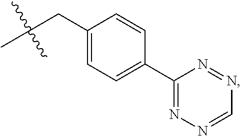 [SMILES: *Cc1ccc(-c2nncnn2)cc1]

unsubstituted or substituted DBCO,

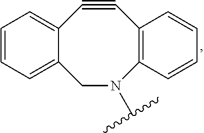 [SMILES: *N1Cc2ccccc2C#Cc2ccccc21]

unsubstituted or substituted BCN,

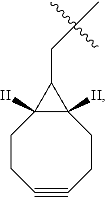

TCO, or

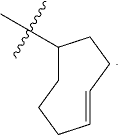 [SMILES: *C1CC/C=C/CCC1]

In some embodiments, the conjugated polymer comprises:

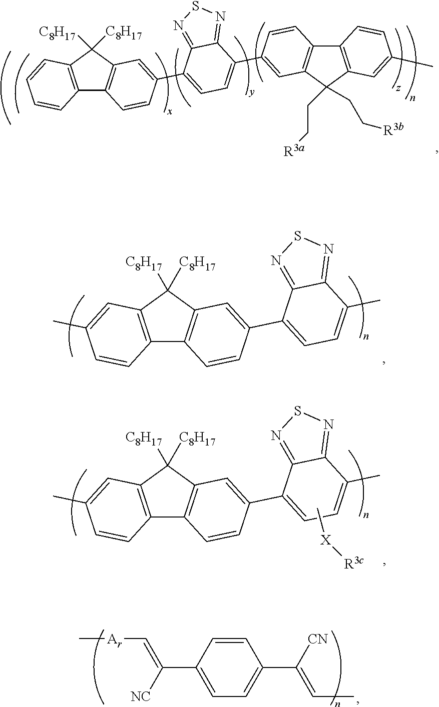

wherein each of R^{3a}, R^{3b} and R^{3c} is independently R^{S} or R^{F};
Aᵣ is

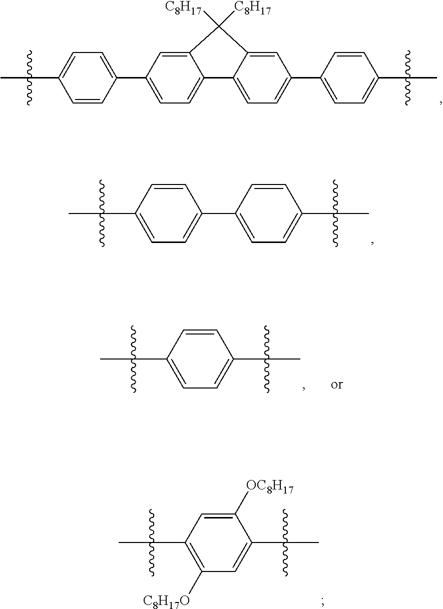

X is O, S, an alkylene linker, or a heteroalkylene linker (such as polyethylene glycol with 2 to 100 repeating units;
R^{F} is azido, amino, a reactive ester,

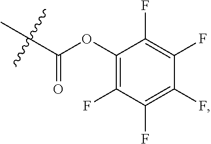 [SMILES: *C(=O)Oc1c(F)c(F)c(F)c(F)c1F]

unsubstituted or substituted 1,2,4,5-tetrazinyl,

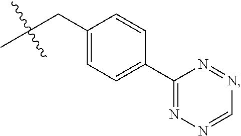 [SMILES: *Cc1ccc(-c2nncnn2)cc1]

unsubstituted or substituted DBCO,

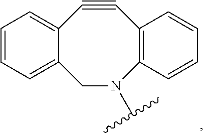 [SMILES: *N1Cc2ccccc2C#Cc2ccccc21]

unsubstituted or substituted BCN,

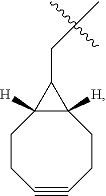

TCO or

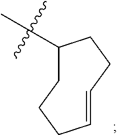 [SMILES: *C1CC/C=C/CCC1]

and
each of m, n, x, y and z is independently an integer from 1 to 100,000.

In some embodiments, the one or more functionalized surfactants comprises an amphiphilic block copolymer or a water-soluble nonionic emulsifier (e.g., Polysorbate 80 (Tween® 80)), or a combination thereof. In some further embodiments, the amphiphilic block copolymer comprises

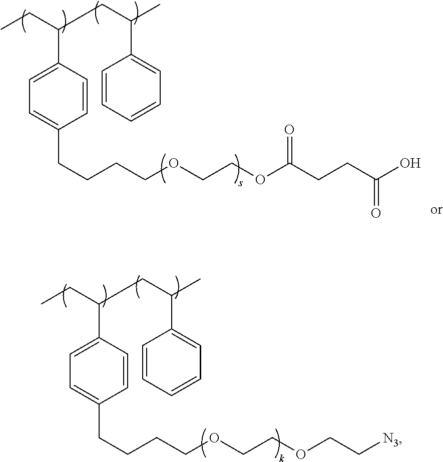

and each of m, n, x, y and z is independently an integer from 1 to 10,000.

In some embodiments, the first labeling reagent comprises a core-shell water-dispersible polymer dot, and wherein the core-shell water-dispersible polymer dot comprises a conjugated polymer nanoparticle core, and a hydrogel shell. In some further embodiments, the core-shell water-dispersible polymer dot further comprises one or more surfactants on the surface of the conjugated polymer nanoparticle core. In yet further embodiments, the one or more surfactants comprises a water-soluble nonionic emulsifier (e.g., Polysorbate 80 (Tween® 80)) or one or more anionic surfactants (e.g., sodium dodecyl sulfate (SDS)), or a combination thereof. In some embodiments, the first labeling reagent comprises a core-shell water-dispersible polymer dot, wherein the core-shell water-dispersible polymer dot comprises a conjugated polymer coated on a supporting particle matrix core. In some embodiments, the conjugated polymer is water-soluble. In other embodiments, the conjugated polymer is hydrophilic, water-swellable. In some such embodiments, the conjugated polymer is coated on the supporting particle matrix core via covalent bonding. In other embodiments, the conjugated polymer is coated on the supporting particle matrix core via non-covalent bonding. In some further embodiments, the functional moiety of the core-shell water-dispersible polymer dot is azido, amino, a reactive ester,

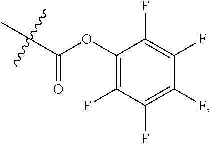 [SMILES: *C(=O)Oc1c(F)c(F)c(F)c(F)c1F]

unsubstituted or substituted 1,2,4,5-tetrazinyl,

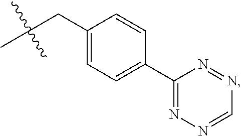 [SMILES: *Cc1ccc(-c2nncnn2)cc1]

unsubstituted or substituted DBCO,

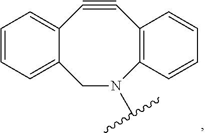 [SMILES: *N1Cc2ccccc2C#Cc2ccccc21]

unsubstituted or substituted BCN,

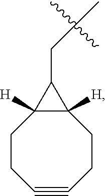

### TCO, or

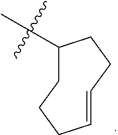 [SMILES: *C1CC/C=C/CCC1]

In some other embodiments, the first reactive moiety of the first type of unlabeled nucleotide is attached to the first labeling reagent via non-covalent interactions. For example, a functional moiety

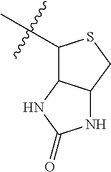 [SMILES: *C1SCC2NC(=O)NC21]

of the unlabeled nucleotide forms noncovalent bonding with an avidin (e.g., streptavidin) moiety of first labeling reagent, or vice versa.

In some embodiments, step (e) also removes the detectable labels of the incorporated nucleotides. In some further embodiments, the detectable labels and the 3′ blocking groups of the incorporated nucleotides are removed in a single chemical reaction.

In some embodiments, the method further comprises (f) washing the solid support with an aqueous wash solution. In some further embodiments, wherein steps (b) to (f) are repeated at least 50, 100, 150, 200, 250, 300, 350, 400, 450, or 500 cycles to determine the target polynucleotide sequences.

In some embodiments, the first labeling reagent is contacted with the extended copy polynucleotides in step (c) by flushing the first labeling reagent through the extended copy polynucleotides. In some embodiments, after step (b) the extended copy polynucleotides on the solid support are washed with a high salt buffer solution prior to contacting with the first labeling reagent. In some embodiments, after step (c) the extended copy polynucleotides on the solid support are washed with a buffer solution prior to imaging the solid support. In some embodiments, the method is performed on an automated sequencing instrument comprising two light sources operating at two different wavelengths.

In some embodiments, incorporation mixture comprises a second type of unlabeled nucleotide having a second reactive moiety. In some embodiments, the second reactive moiety is covalently attached to the second type of unlabeled nucleotide. In some embodiments, the method further comprises contacting the extended copy polynucleotides with a second labeling reagent. The second labeling reagent comprises a second water-soluble conjugated polymer or a second water-dispersible polymer dot comprising a second functional moiety that reacts specifically with the second reactive moiety of the second type of unlabeled nucleotides to form covalent bonding.

### Removal of Labels and Wash Steps

In some embodiments of the sequencing method described herein, step (e) also removes the water-soluble conjugated polymers or water-dispersible polymer dots of the incorporated nucleotides. In some such embodiments, the water-soluble conjugated polymers or water-dispersible polymer dots and the 3′ blocking group are removed in a single step (e.g., under the same chemical reaction condition). In other embodiments, the water-soluble conjugated polymers or water-dispersible polymer dots and the 3′ blocking group are removed in two separate steps (e.g., the water-soluble conjugated polymers or water-dispersible polymer dots and the 3′ blocking group are removed under two separate chemical reaction conditions). In some embodiments, the water-soluble conjugated polymers or water-dispersible polymer dots are removed by cleaving the cleavable linker to which the reactive moiety is covalent attached to the nucleotide. In some further embodiments, the method further comprises (f) washing the solid support with an aqueous wash solution.

In some embodiments, the imaging step (d) is performed by two light sources (e.g., a laser) operating at different wavelengths. In particular, one light source may have a wavelength between about 450 nm to about 460 nm, and the other light source may have a wavelength between about 510 nm to about 540 nm, or between about 520 nm to about 535 nm. As such, step (d) comprises two separate imaging events and two fluorescent measurements.

In further embodiments of the sequencing method described herein, steps (a) through (e) are performed in repeated cycles (e.g., at least 30, 50, 100, 150, 200, 250, 300, 400, or 500 times) and the method further comprises sequentially determining the sequence of at least a portion of the target polynucleotide based on the identity of each sequentially incorporated nucleotide conjugates. In some such embodiments, steps (a) through (e) are repeated at least 50 cycles.

In some embodiments of the method described herein, the incorporation of the nucleotide conjugates is performed by a mutant of 9°N polymerase, such as those disclosed in WO 2005/024010, U.S. Publication Nos. 2020/0131484 A1, 2020/0181587 A1, and 2024/0141427 A1, each of which is incorporated by reference in its entirety. Exemplary polymerases include but not limited to Pol 812, Pol 1901, Pol 1558 or Pol 963. The amino acid sequences of Pol 812, Pol 1901, Pol 1558 or Pol 963 DNA polymerases are described, for example, in U.S. Patent Publication Nos. 2020/0131484 A1 and 2020/0181587 A1.

In some embodiments of the method described herein, the one or more four different types of nucleotides in the incorporation mixture described in step (b) include nucleotide types selected from the group consisting of A, C, G, T and U, and non-natural nucleotide analogs thereof. In further embodiments, the incorporation mixture comprises four different types of nucleotide conjugates (A, C, G, and T or U), or non-natural nucleotide analogs thereof. In further embodiments, the four different types of nucleotide conjugates are dATP, dCTP, dGTP and dTTP or dUTP, or non-natural nucleotide analogs thereof. In further embodiments, each of the four types of nucleotide conjugates in the incorporation mixture contains a 3′ blocking group.

In any embodiments of the method described herein, the density of the immobilized different target polynucleotides (i.e., clusters) on the solid support is about or at least about 50,000/mm², 100,000/mm², 150,000/mm², 200,000/mm², 250,000/mm², 300,000/mm², 350,000/mm², or 400,000/mm².

In some further embodiments, the method is performed on an automated sequencing instrument, and wherein the automated sequencing instrument comprises a single light source operating with a blue laser at about 450 nm to about 460 nm. The incorporation of the first type of the nucleotide is determined by detection in the one of the blue or green channel/region (e.g., at a blue region with a wavelength ranging from about 472 to about 520 nm, or at a green region with a wavelength ranging from about 540 nm to about 640 nm). The incorporation of the second type of nucleotide is determined by detection in the other one of the blue or green detection channel/region. The incorporation of the third type of nucleotide is determined by detection in both the blue and green channels/regions. The incorporation of the fourth type of nucleotide is determined by no detection in either the blue or the green channel/region. Alternatively, the automated sequencing instrument comprises a single light source operating with a green laser at about 500 to about 540 nm, or about 520 nm to about 525 nm. The two emission collection channels are at about 540-585 nm and about 585-640 nm. Detailed disclosures on the method of sequencing utilizes a one-excitation, two-channel detection system (also known as 1Ex-2Ch) are provided in WO 2018/165099 and U.S. Publication No. 2022/0403450 A1, each of which is incorporated by reference in its entirety.

In other embodiments, the automatic sequencing instrument may comprise two light sources operating at different wavelengths (e.g., at 450-460 nm and 520-530 nm). The incorporation of the first type of the nucleotide conjugates is determined by a signal state in the first imaging event and a dark state in the second imaging event. The incorporation of the second type of the nucleotide conjugates is determined by a dark state in the first imaging event and a signal state in the second imaging event. The incorporation of the third type of the nucleotide conjugates is determined by a signal state in both the first imaging event and the second imaging event. The incorporation of the fourth type of the nucleotide conjugates is determined by a dark state in both the first imaging event and the second imaging event.

Though certain reactive moieties of nucleotides and functional moieties of water-soluble conjugated polymers or water-dispersible polymer dots have been discussed in relation to two-tag and three-tag sequencing schemes, other reactive moieties and functional moieties may be used. In further embodiments, the incorporation mixture used in the sequencing method may also include one or more labeled nucleotide(s). For example, the labeled nucleotide(s) may contain a fluorescent label that cannot be excitable by either the first or the second light source.

In any embodiments of methods of sequencing described herein, the method can reduce or eliminate sequence context or sequence specific effect. Two-channel base-calling relies upon the ability to discriminate bases by their intensity in two emission color channels. This intensity modulation adds noise to the system and can cause miscalls when the sequence-specific intensity modulation shift a given cluster's intensity towards a decision boundary. Using a single ffN (such as ffA) which is subsequently conjugated with water-soluble conjugated polymers or water-dispersible polymer dots that can produce color in two channels reduce or eliminate sequence-specific intensity shifts and thereby improve base calling accuracy.

### General Description on Sequencing by Synthesis

In some embodiments, a synthetic step is carried out and may optionally comprise incubating a template or target polynucleotide strand with a reaction mixture comprising fluorescently labeled nucleotides of the disclosure. A polymerase can also be provided under conditions which permit formation of a phosphodiester linkage between a free 3′ hydroxy group on a polynucleotide strand annealed to the template or target polynucleotide strand and a 5′ phosphate group on the labeled nucleotide. Thus, a synthetic step can include formation of a polynucleotide strand as directed by complementary base pairing of nucleotides to a template/target strand.

In all embodiments of the methods, the detection step may be carried out while the polynucleotide strand into which the labeled nucleotides are incorporated is annealed to a template/target strand, or after a denaturation step in which the two strands are separated. Further steps, for example chemical or enzymatic reaction steps or purification steps, may be included between the synthetic step and the detection step. In particular, the polynucleotide strand incorporating the labeled nucleotide(s) may be isolated or purified and then processed further or used in a subsequent analysis. By way of example, polynucleotide strand incorporating the labeled nucleotide(s) as described herein in a synthetic step may be subsequently used as labeled probes or primers. In other embodiments, the product of the synthetic step set forth herein may be subject to further reaction steps and, if desired, the product of these subsequent steps purified or isolated.

Suitable conditions for the synthetic step will be well known to those familiar with standard molecular biology techniques. In one embodiment, a synthetic step may be analogous to a standard primer extension reaction using nucleotide precursors, including the labeled nucleotides as described herein, to form an extended polynucleotide strand (primer polynucleotide strand) complementary to the template/target strand in the presence of a suitable polymerase enzyme. In other embodiments, the synthetic step may itself form part of an amplification reaction producing a labeled double stranded amplification product comprised of annealed complementary strands derived from copying of the primer and template polynucleotide strands. Other exemplary synthetic steps include nick translation, strand displacement polymerization, random primed DNA labeling, etc. A particularly useful polymerase enzyme for a synthetic step is one that is capable of catalyzing the incorporation of the labeled nucleotides as set forth herein. A variety of naturally occurring or mutant/modified polymerases can be used. By way of example, a thermostable polymerase can be used for a synthetic reaction that is carried out using thermocycling conditions, whereas a thermostable polymerase may not be desired for isothermal primer extension reactions. Suitable thermostable polymerases which are capable of incorporating the labeled nucleotides according to the disclosure include those described in WO 2005/024010 or WO06120433, each of which is incorporated herein by reference. In synthetic reactions which are carried out at lower temperatures such as 37° C., polymerase enzymes need not necessarily be thermostable polymerases, therefore the choice of polymerase will depend on a number of factors such as reaction temperature, pH, strand-displacing activity and the like.

In specific non-limiting embodiments, the disclosure encompasses methods of nucleic acid sequencing, re-sequencing, whole genome sequencing, single nucleotide polymorphism scoring, any other application involving the detection of the modified nucleotide or nucleoside labeled with dyes set forth herein when incorporated into a polynucleotide.

SBS generally involves sequential addition of one or more nucleotides or oligonucleotides to a growing polynucleotide chain in the 5′ to 3′ direction using a polymerase or ligase in order to form an extended polynucleotide chain complementary to the template/target nucleic acid to be sequenced. The identity of the base present in one or more of the added nucleotide(s) can be determined in a detection or “imaging” step. The identity of the added base may be determined after each nucleotide incorporation step. The sequence of the template may then be inferred using conventional Watson-Crick base-pairing rules. The use of the nucleotides labeled with dyes set forth herein for determination of the identity of a single base may be useful, for example, in the scoring of single nucleotide polymorphisms, and such single base extension reactions are within the scope of this disclosure.

In an embodiment of the present disclosure, the sequence of a template/target polynucleotide is determined by detecting the incorporation of one or more nucleotides into a nascent strand complementary to the template polynucleotide to be sequenced through the detection of fluorescent label(s) attached to the incorporated nucleotide(s). Sequencing of the template polynucleotide can be primed with a suitable primer (or prepared as a hairpin construct which will contain the primer as part of the hairpin), and the nascent chain is extended in a stepwise manner by addition of nucleotides to the 3′ end of the primer in a polymerase-catalyzed reaction.

In particular embodiments, each of the different nucleotide triphosphates (A, T, G and C) may be labeled with a unique fluorophore and also comprises a blocking group at the 3′ position to prevent uncontrolled polymerization. Alternatively, one of the four nucleotides may be unlabeled (dark). The polymerase enzyme incorporates a nucleotide into the nascent chain complementary to the template/target polynucleotide, and the blocking group prevents further incorporation of nucleotides. Any unincorporated nucleotides can be washed away and the fluorescent signal from each incorporated nucleotide can be “read” optically by suitable means, such as a charge-coupled device using light source excitation and suitable emission filters. The 3′-blocking group and fluorescent dye compounds can then be removed (deprotected) (simultaneously or sequentially) to expose the nascent chain for further nucleotide incorporation.

The method, as exemplified above, utilizes the incorporation of fluorescently labeled, 3′ blocked nucleotides A, G, C, and T into a growing strand complementary to the immobilized polynucleotide, in the presence of DNA polymerase. The polymerase incorporates a base complementary to the target polynucleotide but is prevented from further addition by the 3′ blocking group. The label of the incorporated nucleotide can then be determined, and the blocking group removed by chemical cleavage to allow further polymerization to occur. The nucleic acid template to be sequenced in a SBS reaction may be any polynucleotide that it is desired to sequence. The nucleic acid template for a sequencing reaction will typically comprise a double stranded region having a free 3′ hydroxy group that serves as a primer or initiation point for the addition of further nucleotides in the sequencing reaction. The region of the template to be sequenced will overhang this free 3′ hydroxy group on the complementary strand. The overhanging region of the template to be sequenced may be single stranded but can be double-stranded, provided that a “nick” is present on the strand complementary to the template strand to be sequenced to provide a free 3′ OH group for initiation of the sequencing reaction. In such embodiments, sequencing may proceed by strand displacement. In certain embodiments, a primer bearing the free 3′ hydroxy group may be added as a separate component (e.g., a short oligonucleotide) that hybridizes to a single-stranded region of the template to be sequenced. Alternatively, the primer and the template strand to be sequenced may each form part of a partially self-complementary nucleic acid strand capable of forming an intra-molecular duplex, such as for example a hairpin loop structure. Hairpin polynucleotides and methods by which they may be attached to solid supports are disclosed in PCT Publication Nos. WO0157248 and WO2005/047301, each of which is incorporated herein by reference. Nucleotides can be added successively to a growing primer, resulting in synthesis of a polynucleotide chain in the 5′ to 3′ direction. The nature of the base which has been added may be determined, particularly but not necessarily after each nucleotide addition, thus providing sequence information for the nucleic acid template. Thus, a nucleotide is incorporated into a nucleic acid strand (or polynucleotide) by joining of the nucleotide to the free 3′ hydroxy group of the nucleic acid strand via formation of a phosphodiester linkage with the 5′ phosphate group of the nucleotide.

The nucleic acid template to be sequenced may be DNA or RNA, or even a hybrid molecule comprised of deoxynucleotides and ribonucleotides. The nucleic acid template may comprise naturally occurring and/or non-naturally occurring nucleotides and natural or non-natural backbone linkages, provided that these do not prevent copying of the template in the sequencing reaction.

In certain embodiments, the nucleic acid template to be sequenced may be attached to a solid support via any suitable linkage method known in the art, for example via covalent attachment. In certain embodiments template polynucleotides may be attached directly to a solid support (e.g., a silica-based support). However, in other embodiments of the disclosure the surface of the solid support may be modified in some way so as to allow either direct covalent attachment of template polynucleotides, or to immobilize the template polynucleotides through a hydrogel or polyelectrolyte multilayer, which may itself be non-covalently attached to the solid support.

Arrays in which polynucleotides have been directly attached to a support (for example, silica-based supports such as those disclosed in WO00/06770 (incorporated herein by reference), wherein polynucleotides are immobilized on a glass support by reaction between a pendant epoxide group on the glass with an internal amino group on the polynucleotide. In addition, polynucleotides can be attached to a solid support by reaction of a sulfur-based nucleophile with the solid support, for example, as described in WO2005/047301 (incorporated herein by reference). A still further example of solid-supported template polynucleotides is where the template polynucleotides are attached to hydrogel supported upon silica-based or other solid supports, for example, as described in WO00/31148, WO01/01143, WO02/12566, WO03/014392, U.S. Pat. No. 6,465,178 and WO00/53812, each of which is incorporated herein by reference.

A particular surface to which template polynucleotides may be immobilized is a polyacrylamide hydrogel. Polyacrylamide hydrogels are described in the references cited above and in WO2005/065814, which is incorporated herein by reference. Specific hydrogels that may be used include those described in WO2005/065814 and U.S. Pub. No. 2014/0079923. In one embodiment, the hydrogel is PAZAM (poly(N-(5-azidoacetamidylpentyl) acrylamide-co-acrylamide)).

DNA template molecules can be attached to beads or microparticles, for example, as described in U.S. Pat. No. 6,172,218 (which is incorporated herein by reference). Attachment to beads or microparticles can be useful for sequencing applications. Bead libraries can be prepared where each bead contains different DNA sequences. Exemplary libraries and methods for their creation are described in Nature, 437, 376-380 (2005); Science, 309, 5741, 1728-1732 (2005), each of which is incorporated herein by reference. Sequencing of arrays of such beads using nucleotides set forth herein is within the scope of the disclosure.

Template(s) that are to be sequenced may form part of an “array” on a solid support, in which case the array may take any convenient form. Thus, the method of the disclosure is applicable to all types of high-density arrays, including single-molecule arrays, clustered arrays, and bead arrays. Nucleotides labeled with dye compounds of the present disclosure may be used for sequencing templates on essentially any type of array, including but not limited to those formed by immobilization of nucleic acid molecules on a solid support.

However, nucleotides labeled with dye compounds of the disclosure are particularly advantageous in the context of sequencing of clustered arrays. In clustered arrays, distinct regions on the array (often referred to as sites, or features) comprise multiple polynucleotide template molecules. Generally, the multiple polynucleotide molecules are not individually resolvable by optical means and are instead detected as an ensemble. Depending on how the array is formed, each site on the array may comprise multiple copies of one individual polynucleotide molecule (e.g., the site is homogenous for a particular single- or double-stranded nucleic acid species) or even multiple copies of a small number of different polynucleotide molecules (e.g., multiple copies of two different nucleic acid species). Clustered arrays of nucleic acid molecules may be produced using techniques generally known in the art. By way of example, WO 98/44151 and WO00/18957, each of which is incorporated herein, describe methods of amplification of nucleic acids wherein both the template and amplification products remain immobilized on a solid support in order to form arrays comprised of clusters or “colonies” of immobilized nucleic acid molecules. The nucleic acid molecules present on the clustered arrays prepared according to these methods are suitable templates for sequencing using nucleotides labeled with dye compounds of the disclosure.

Nucleotides labeled with dye compounds of the present disclosure are also useful in sequencing of templates on single molecule arrays. The term “single molecule array” or “SMA” as used herein refers to a population of polynucleotide molecules, distributed (or arrayed) over a solid support, wherein the spacing of any individual polynucleotide from all others of the population is such that it is possible to individually resolve the individual polynucleotide molecules. The target nucleic acid molecules immobilized onto the surface of the solid support can thus be capable of being resolved by optical means in some embodiments. This means that one or more distinct signals, each representing one polynucleotide, will occur within the resolvable area of the particular imaging device used.

Single molecule detection may be achieved wherein the spacing between adjacent polynucleotide molecules on an array is at least 100 nm, more particularly at least 250 nm, still more particularly at least 300 nm, even more particularly at least 350 nm. Thus, each molecule is individually resolvable and detectable as a single molecule fluorescent point, and fluorescence from said single molecule fluorescent point also exhibits single step photobleaching.

The terms “individually resolved” and “individual resolution” are used herein to specify that, when visualized, it is possible to distinguish one molecule on the array from its neighboring molecules. Separation between individual molecules on the array will be determined, in part, by the particular technique used to resolve the individual molecules. The general features of single molecule arrays will be understood by reference to published applications WO00/06770 and WO01/57248, each of which is incorporated herein by reference. Although one use of the labeled nucleotides of the disclosure is in SBS reactions, the utility of such nucleotides is not limited to such methods. In fact, the labeled nucleotides described herein may be used advantageously in any sequencing methodology which requires detection of fluorescent labels attached to nucleotides incorporated into a polynucleotide.

In particular, nucleotides labeled with dye compounds of the disclosure may be used in automated fluorescent sequencing protocols, particularly fluorescent dye-terminator cycle sequencing based on the chain termination sequencing method of Sanger and co-workers. Such methods generally use enzymes and cycle sequencing to incorporate fluorescently labeled dideoxynucleotides in a primer extension sequencing reaction. So-called Sanger sequencing methods, and related protocols (Sanger-type), utilize randomized chain termination with labeled dideoxynucleotides.

### EXAMPLES

Additional embodiments are disclosed in further detail in the following examples, which are not in any way intended to limit the scope of the claims.

### Example 1. Synthesis of a Water-Dispersible Polymer Dot

### Synthesis of CP—F

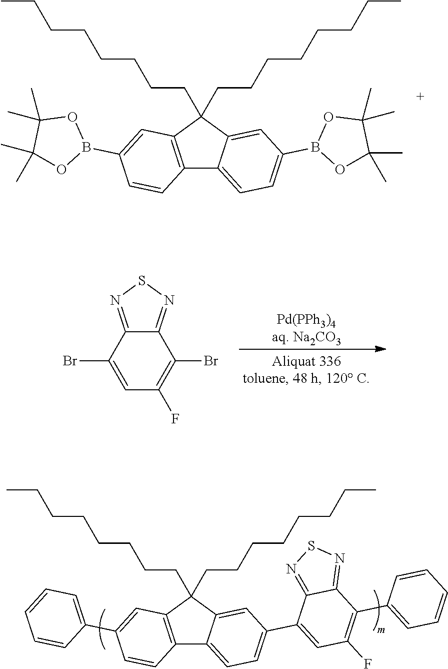

9, 9-Dioctyl-9H-fluorene-2, 7-diboronic acid bis (pinacol) ester (501.0 mg, 0.780 mmol), 4,7-dibromo-5-fluoro-2, 1,3-benzothiadiazole (243.2 mg, 0.780 mmol), Pd(PPh₃)₄ (18.0 mg, 0.016 mmol) and Aliquot 336 (1 drop) were added to a high pressure vial and degassed with argon. Degassed toluene (6.5 mL) and degassed aqueous Na₂CO₃ (2 M, 4 mL) were added and the mixture was heated at 120° C. for 48 h. To end-cap the polymer, 1 mL of a solution of phenylboronic acid pinacol ester (0.1 mg) and Pd(PPh₃)₄ (1.00 mg) in degassed toluene (0.5 mL) were added and the resulting mixture was stirred for 1 h at 120° C. 0.1 mL of a solution of bromobenzene (0.1 mL) in degassed toluene (0.5 mL) were subsequently added and stirring at 120° C. was maintained for a further 1 h. The mixture was cooled to room temperature, precipitated into methanol and filtered through a Soxhlet thimble. The polymer was purified by Soxhlet extraction with hexane, methanol, acetone and chloroform under argon. The chloroform fraction was concentrated under vacuum and precipitated into methanol to give CP—F as a yellow solid (264 mg, 63%). ¹H NMR (400 MHZ, CDCl₃) δ 8.24-7.75 (m, 7H), 2.13 (m, 4H), 1.71-0.81 (m, 30H).

### Synthesis of CP-PEG36-N₃

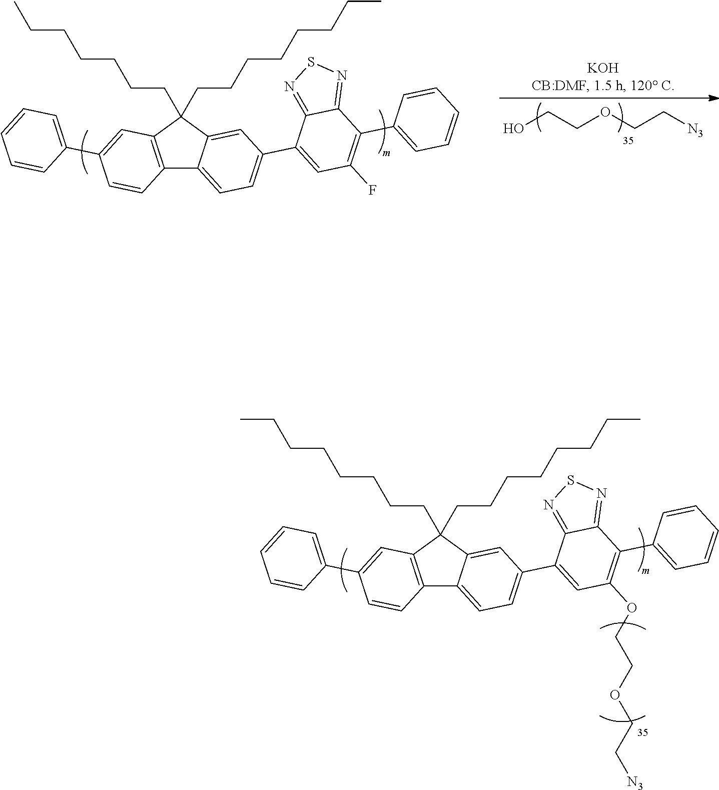

CP—F (10.0 mg) and KOH (40.0 mg, 0.713 mmol) were suspended in a mixture of degassed anhydrous chlorobenzene (1.5 mL) and degassed anhydrous dimethylformamide (0.5 mL). After heating to 120° C. for 30 min, a solution of azido-PEG36-alcohol (36.15 mg, 0.022 mmol) in degassed dimethylformamide (0.5 mL) was added and the resulting mixture was heated to 120° C. for a further 1 h. After allowing to cool to room temperature, the solvent was removed under vacuum. The resulting solid was successively washed with water (100 mL) and dried under vacuum to give the title product as a yellow solid (21.2 mg, 53%). ¹H NMR (400 MHZ, CDCl₃) δ 8.09-7.84 (m, 7H), 4.35 (br s, 2H), 3.81 (m, 2H), 3.73-3.45 (m, 138H), 3.38 (m, 2H), 2.07 (br s, 4H), 1.20-0.79 (m, 30H).

### Polymer Dot Fabrication

CP-PEG36-N₃ was dissolved in inhibitor-free tetrahydrofuran (500 μg/mL) with sonication for 3 min and filtered through a 0.45 μm PTFE syringe filter. To fabricate the nanoparticles, 8 mL of the resulting CP-PEG36-N₃ solution were rapidly injected to 80 mL of distilled water, followed by sonication using a sonicator probe for 3 min. The tetrahydrofuran was removed under a stream of nitrogen and the nanoparticle suspension was filtered through a 0.2 μm nylon syringe filter. The samples were concentrated to various degrees using centrifugal filters with 30 kDa molecular weight cut-off.

### Example 2. Synthesis of a Water-Dispersible Polymer Dot Via Knoevenagel Condensation and Emulsion Polymerization

### Knoevenagel Condensation

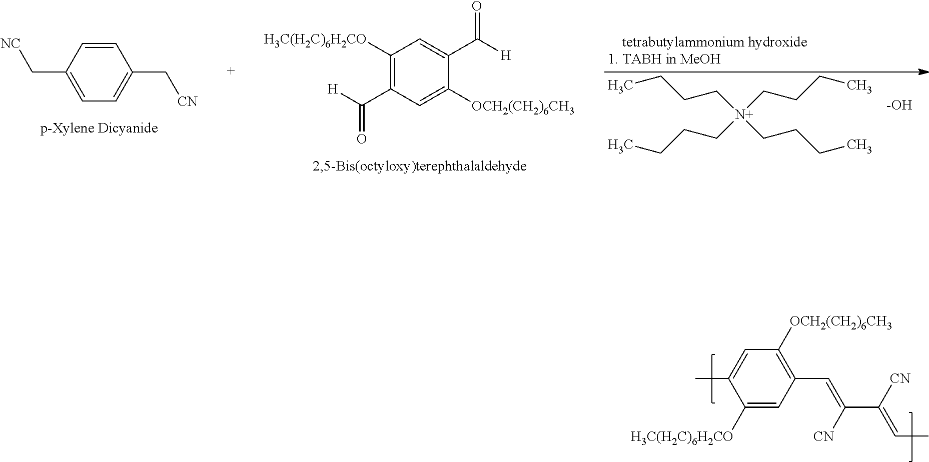

### Emulsion Polymerization

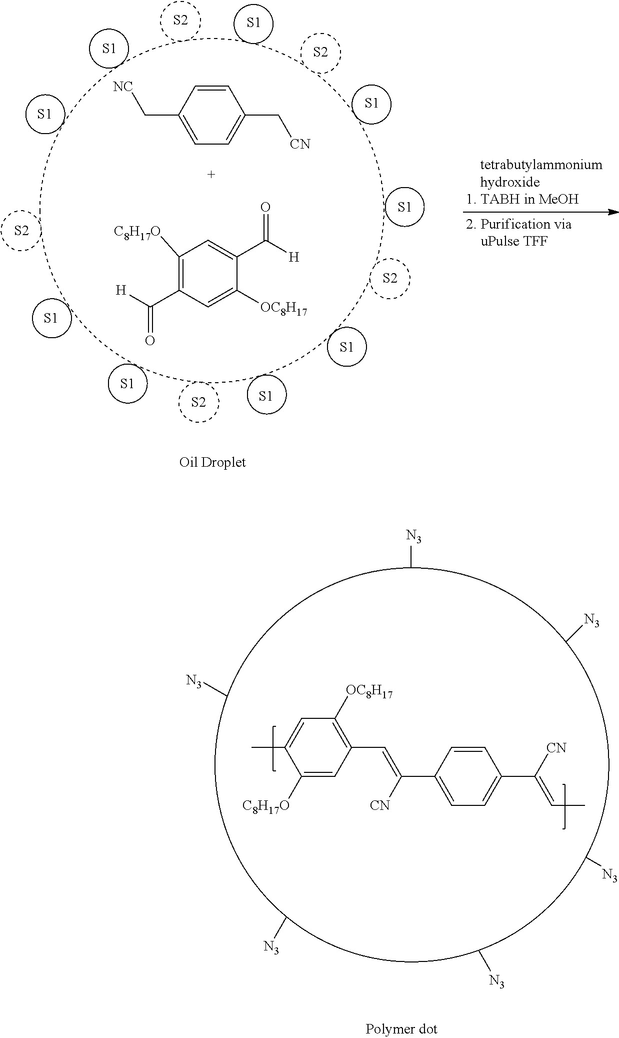

Surfactant S1 is Tween 80; Surfactant S2 is a polystylene based copolymer having the structure:

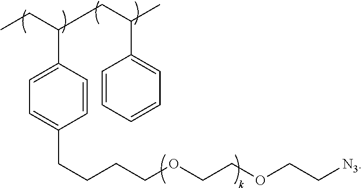

The synthesis of the polymer dot dispersion was conducted via a one-pot synthesis. Tween80 (1.696 g, 1.295 mM) and azide containing polystyrene-g-poly(ethylene oxide) copolymers were mixed at 60° C. in a 25 mL RBF for 1 hour prior to addition of the monomer. 2,5-bis(octyloxy)terephthalaldehyde) (0.044 g, 0.112 mM) and p-xylene dicyanide (0.018 g, 0.112 mM) were then added and dissolved in co-surfactant mixture at 60° C. for 30 min. Deionized water (15 mL) is then added at 50° C. in 90 min with a constant rate 0.17 mL/min via a syringe pump. A clear dispersion should be obtained, which is then cooled down to RT in 30 min with constant stirring. Tetrabutylammonium hydroxide (0.6 mL of TBAH 1.0 M in EtOH) was then added to the dispersion and the polymerization is conducted for 14 h at RT.

Characterization: The polymer dot dispersion was purified using a μ-Pulse tangential flow filtration (TFF) (Formulatrix Co.) with a 100 k Da cut-off membrane chip. The particle sizes were measured using Dynamic Light Scattering (Malvern Zetasizer Nano). The absorption and emission spectra were recording using a UV-Vis instrument (Carry) and a fluorescence spectrometer (FLS1000, Edinburgh Instrument).

Materials: azide containing polystyrene-g-poly(ethylene oxide) copolymers (Mn=14 kDa, 10 PEO branches) were purchased from PolymerSource (Canada). Tween80 (high purity rate), 2,5-bis(octyloxy)terephthalaldehyde) (98%) and tetrabutylammonium hydroxide (TBAH 1.0 M in EtOH) were purchased from Sigma (UK). p-Xylene dicyanide was purchased from TCI Chemicals (UK).

The azido functionalized polymer dot further reacted with a DBCO reagent to provide a final polymer dot with methyl tetrazine functionality. The polymer dot was purified by TFF with about 30 kDa molecular weight cut off (MWCO).

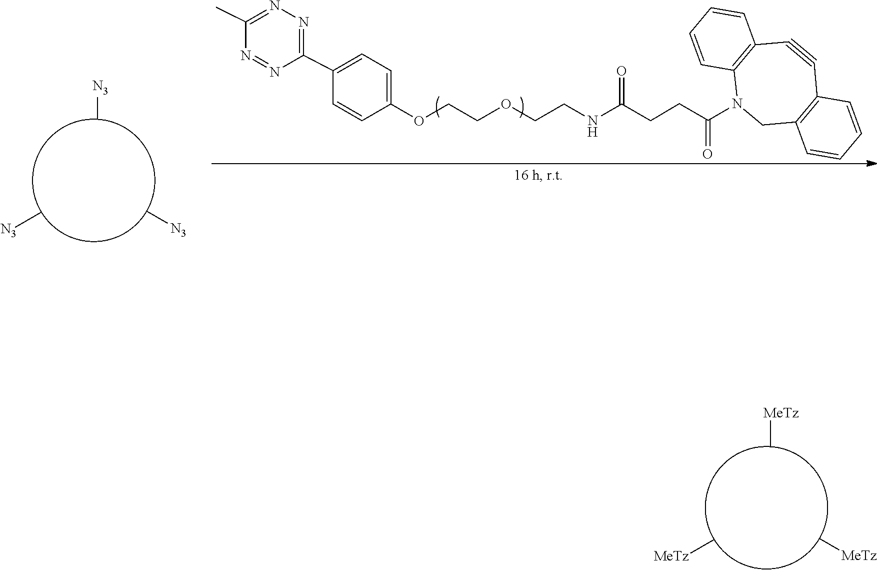

### Example 3. Two-Channel Sequencing by Synthesis Using a Water-Dispersible Polymer Dot as a Post Incorporation Labeling Reagent

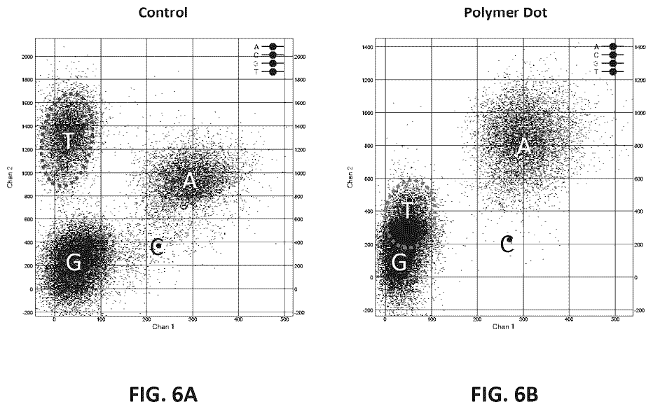

In this experiment, two SBS runs were conducted on Illumina Miseq® instrument with blue and green lasers operating at 460 nm and 532 nm. Images were taken simultaneously through two separate collection channels. The standard incorporation mixture include: (1) a set of nucleotides comprising dark ffG, dark ffT, ffC-db-AOL-NR550S0 (a known green dye), ffA-db-AOL-NR550S0 and ffA-db-AOL-coumarin Dye A; (2) DNA polymerase and (3) a glycine buffer. All ffNs are blocked with 3′ AOM blocking group. The resulting scatterplot is shown in FIG. 6A.

Coumarin dye A has strong fluorescence and great stability. This dye is disclosed in U.S. Publication No. 2020/0277670 A1 (incorporated by reference), having the structure moiety

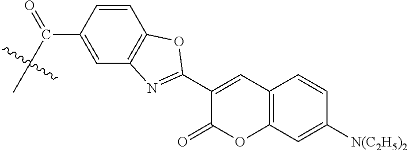

when conjugated with the ffA.

A second incorporation mix includes (1) a set of nucleotides comprising dark ffG, dark ffT, ffC-db-AOL-AOL-TCO, ffA-db-AOL-coumarin Dye A; ffA-db-AOL-NR550S0 (2) DNA polymerase and (3) a glycine buffer. All ffNs are blocked with 3′ AOM blocking group. The post-incorporation reagent contains methyl tetrazine-functionalized polymer dots (described in Example 2) dispersed in a hybridization buffer. The post incorporation mixture was flushed through the incorporated ffNs at 60° C. The resulting scatterplot is showing in FIG. 6B.

1. A nucleotide, oligonucleotide or polynucleotide comprising a water-soluble conjugated polymer or a water-dispersible polymer dot attached thereto as a detectable label.

2. The nucleotide, oligonucleotide or polynucleotide of claim 1, wherein the water-soluble conjugated polymer comprise one or more repeating unit moieties selected from the group consisting of: 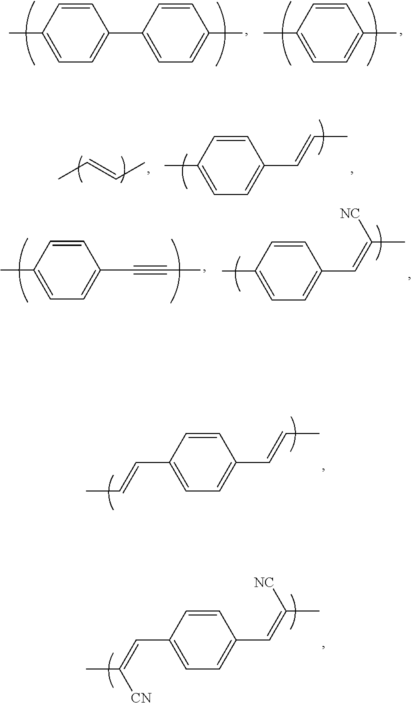
a five membered heteroarylene containing one, two or three heteroatoms selected from the group consisting of N, O and S, 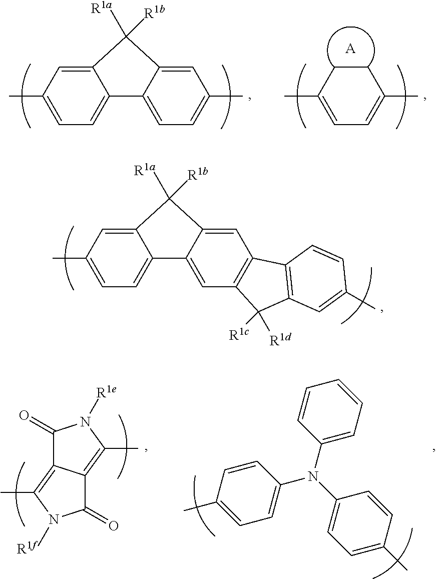
and combinations thereof;
wherein each of R^{1a}, R^{1b}, R^{1c}, R^{1d}, R^{1e} and R^{1f} is independently H, unsubstituted C₁-C₁₂ alkyl, C₁-C₁₂ alkyl substituted with one or more water solubilizing groups, or 2 to 20 membered heteroalkyl;
ring A is a five or six membered heteroaryl containing one, two or three heteroatoms selected from the group consisting of N, O and S, and
wherein each structure is optionally substituted with one or more electron donating groups, one or more electron withdrawing groups, one or more functional moiety R^{F}, or one or more water solubilizing group R^{S}, or combinations thereof.

3. The nucleotide, oligonucleotide or polynucleotide of claim 2, wherein the water solubilizing groups are selected from the group consisting of phosphate; 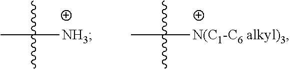
wherein each C₁-C₆ alkyl is optionally substituted with one or more substituents selected from phosphate,

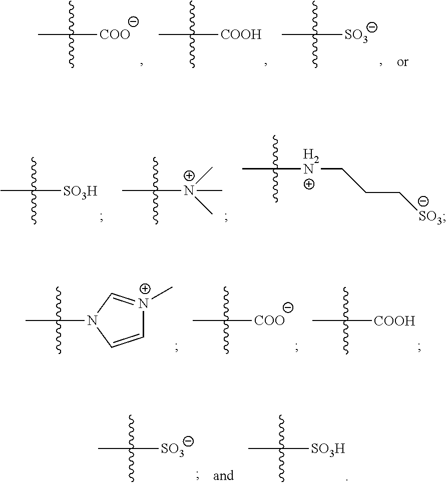

4. The nucleotide, oligonucleotide or polynucleotide of claim 2, wherein the conjugated polymer is covalently bounded to the nucleotide, oligonucleotide or polynucleotide through reaction of the functional moiety R^{F} of the conjugated polymer with a reactive moiety of the oligonucleotide or polynucleotide.

5. The nucleotide, oligonucleotide or polynucleotide of claim 4, wherein the conjugated polymer comprises the structure: 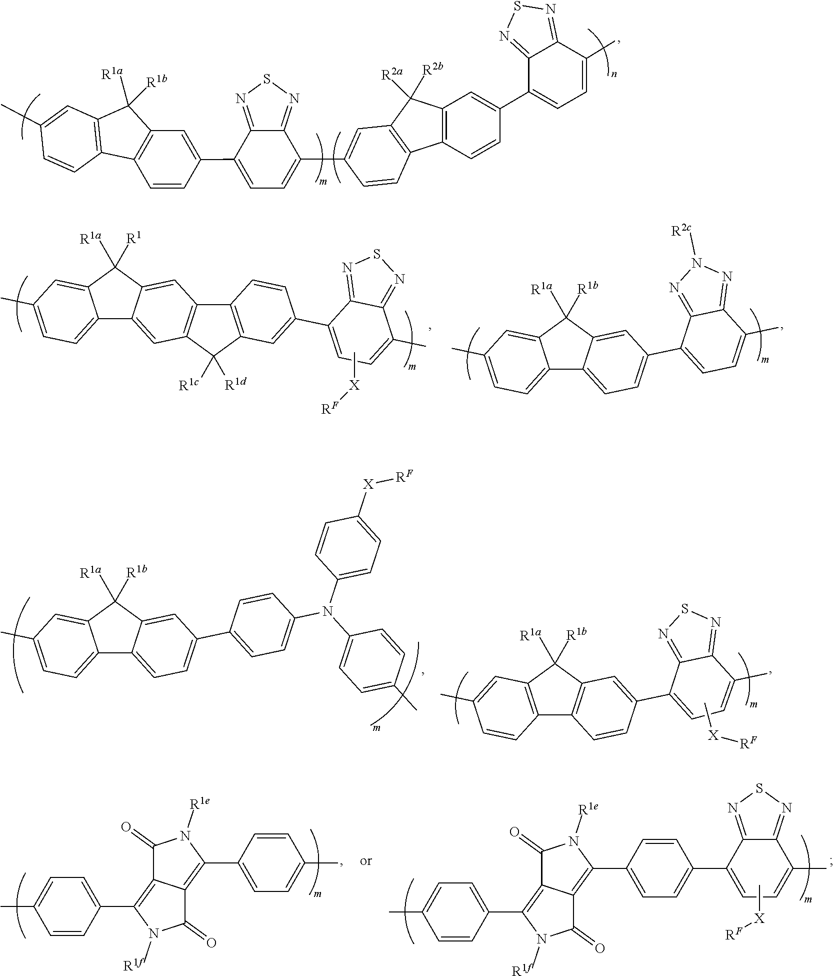
wherein each of R^{2a}, R^{2b} and R^{2c} is independently H, R^{F} or C₁-C₁₂ alkyl substituted with R^{F};
X is O, S, an alkylene linker, or a heteroalkylene linker;
each R^{F} is independently azido, amino, a reactive ester, 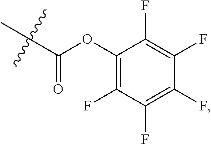 [SMILES: *C(=O)Oc1c(F)c(F)c(F)c(F)c1F]
unsubstituted or substituted 1,2,4,5-tetrazinyl, 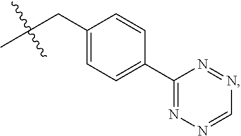 [SMILES: *Cc1ccc(-c2nncnn2)cc1]
unsubstituted or substituted dibenzocyclooctyne (DBCO), 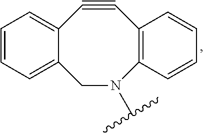 [SMILES: *N1Cc2ccccc2C#Cc2ccccc21]
unsubstituted or substituted bicyclo[6.1.0]nonynyl (BCN), 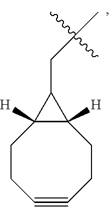 [SMILES: *CC1[C@H]2CCC#CCC[C@@H]12]
unsubstituted or substituted transcyclooctenyl (TCO), or 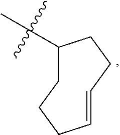 [SMILES: *C1CC/C=C/CCC1]
and
each of m and n is independently an integer from 1 to 100,000.

6. The nucleotide, oligonucleotide or polynucleotide of claim 1, comprising the water-dispersible polymer dot covalently bounded thereto, wherein the water-dispersible polymer dot comprises a conjugated polymer nanoparticle and one or more functionalized surfactants attached to the surface of the nanoparticle.

7.-9. (canceled)

10. The nucleotide, oligonucleotide or polynucleotide of claim 6, wherein the water-dispersible polymer dot is covalently bounded to the nucleotide, oligonucleotide or polynucleotide through reaction of a functional moiety of the conjugated polymer or a functional moiety of the one or more functionalized surfactants with a reactive moiety of the nucleotide, oligonucleotide or polynucleotide.

11. The nucleotide, oligonucleotide or polynucleotide of claim 10, wherein the functional moiety of the conjugated polymer or one or more functionalized surfactants is selected from the group consisting of azido, amino, a reactive ester, 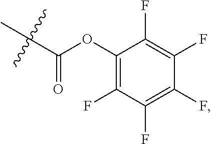 [SMILES: *C(=O)Oc1c(F)c(F)c(F)c(F)c1F]
unsubstituted or substituted 1,2,4,5-tetrazinyl, 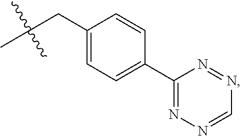 [SMILES: *Cc1ccc(-c2nncnn2)cc1]
unsubstituted or substituted DBCO, 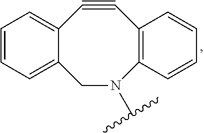 [SMILES: *N1Cc2ccccc2C#Cc2ccccc21]
unsubstituted or substituted BCN, 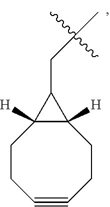 [SMILES: *CC1[C@H]2CCC#CCC[C@@H]12]
unsubstituted or substituted TCO, and

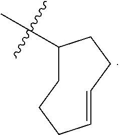 [SMILES: *C1CC/C=C/CCC1]

12. The nucleotide, oligonucleotide or polynucleotide of claim 10, wherein the one or more functionalized surfactants comprises an amphiphilic block copolymer or a water soluble nonionic emulsifier (e.g., Polysorbate 80 (Tween® 80)), or a combination thereof.

13. The nucleotide, oligonucleotide or polynucleotide of claim 12, wherein the amphiphilic block copolymer comprises 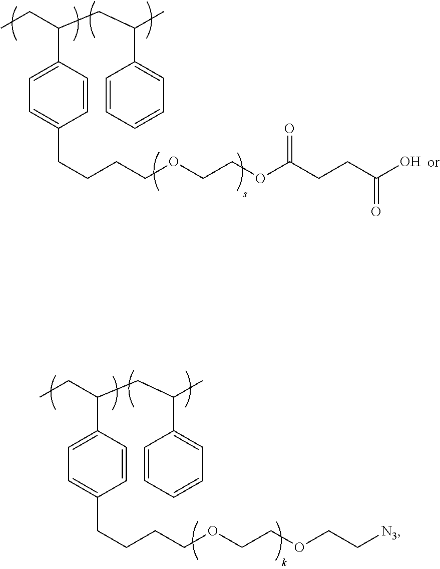
each of s and k is independently an integer from 1 to 10,000.

14. The nucleotide, oligonucleotide or polynucleotide of claim 1, comprising a core-shell water-dispersible polymer dot covalently bounded thereto, wherein the core-shell water-dispersible polymer dot comprises a conjugated polymer nanoparticle core, and a hydrogel shell.

15. The nucleotide, oligonucleotide or polynucleotide of claim 14, wherein the core-shell water-dispersible polymer dot further comprises one or more surfactants on the surface of the conjugated polymer nanoparticle core.

16. The nucleotide, oligonucleotide or polynucleotide of claim 15, wherein the one or more surfactants comprises a water soluble nonionic emulsifier (e.g., Polysorbate 80 (Tween® 80)) or an anionic surfactant (e.g., sodium dodecyl sulfate (SDS)), or a combination thereof.

17. The nucleotide, oligonucleotide or polynucleotide of claim 14, wherein the core-shell water-dispersible polymer dot is covalently bounded to the oligonucleotide or polynucleotide through reaction of a functional moiety of the hydrogel shell with a reactive moiety of the nucleotide, oligonucleotide or polynucleotide.

18. (canceled)

19. The nucleotide, oligonucleotide or polynucleotide of claim 14, wherein the hydrogel shell comprises an acrylamide hydrogel.

20. (canceled)

21. The nucleotide, oligonucleotide or polynucleotide of claim 1, comprising a core-shell water-dispersible polymer dot covalently bounded thereto, wherein the core-shell water-dispersible polymer dot comprises a conjugated polymer coated on a supporting particle matrix core, and wherein the conjugated polymer is water-soluble, water-swellable, or hydrophilic.

22. The nucleotide, oligonucleotide or polynucleotide of claim 21, wherein the core-shell water-dispersible polymer dot is covalently bounded to the nucleotide, oligonucleotide or polynucleotide through reaction of a functional moiety of the water-soluble conjugated polymer or the supporting particle matrix with a reactive moiety of the nucleotide, oligonucleotide or polynucleotide.

23.-24. (canceled)

25. The nucleotide, oligonucleotide or polynucleotide of claim 1, wherein the water-soluble conjugated polymer or the water-dispersible polymer dot is covalently bounded to an unlabeled nucleotide, or an unlabeled terminal nucleotide moiety of the oligonucleotide or polynucleotide via a cleavable linker.

26. (canceled)

27. The nucleotide, oligonucleotide or polynucleotide of claim 1, wherein the oligonucleotide or polynucleotide is at least partially hybridized to a template polynucleotide immobilized on a solid support.

28. (canceled)

29. A kit of sequencing application, comprising:
an incorporation mixture composition comprising one or more of four different types of nucleotides each comprising a 3′ blocking group, wherein a first type of nucleotide is the nucleotide of claim 1.

30. A kit for sequencing application, comprising:
an incorporation mixture composition comprising one or more of four different types of nucleotides each comprising a 3′ blocking group, wherein a first type of unlabeled nucleotide comprises a first reactive moiety covalently attached thereto; and
a first labeling reagent comprising a water-soluble conjugated polymer or a water-dispersible polymer dot, each having a first functional moiety that is capable of reacting specifically with the first reactive moiety of the first type of unlabeled nucleotide to form covalent or non-covalent bonding.

31.-47. (canceled)

48. A method of determining the sequences of a plurality of different target polynucleotides, comprising:
(a) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(b) contacting the solid support with an aqueous solution comprising DNA polymerase and one or more of four different types of unlabeled nucleotides under conditions suitable for DNA polymerase-mediated primer extension, and incorporating one type of nucleotides into the sequencing primers to produce extended copy polynucleotides; wherein
each of the four types of nucleotides comprises a 3′ blocking group; and
the incorporation mixture comprises a first type of unlabeled nucleotide having a first reactive moiety covalently attached to the first type of unlabeled nucleotide;
(c) contacting the extended copy polynucleotides with a first labeling reagent comprising a water-soluble conjugated polymer or a water-dispersible polymer dot comprising a functional moiety that reacts specifically with the first reactive moiety of the first type of unlabeled nucleotides to form covalent or non-covalent bonding;
(d) imaging the solid support and performing one or more fluorescent measurements of the extended copy polynucleotides; and
(e) removing the 3′ blocking group of the incorporated nucleotides.

49.-72. (canceled)
